SEQUENCE LISTING (46 sequences)

SEQ ID NO: 1 - protein, 316 aa, Xenopus laevis
AWSVNNFLMTGPKAYLTYSASVAVGAQNGIEECKYQFAWERWNCPESTLQLATHNGLRSATRETSFVHAISSAGVMYTLTRNCSMGDFDNCGCDDSRNGRIGGRGWVWGGCSDNAEFGERISKLFVDGLETGQDARALMNLHNNEAGRLAVKETMKRTCKCHGISGSCSIQTCWLQLAEFRDIGNHLKIKHDQALKLEMDKRKMRSGNSADNRGAIADAFSSVAGSELIFLEDSPDYCLKNISLGLQGTEGRECLQSGKNLSQWERRSCKRLCTDCGLRVEEKKTEIISSCNCKFHWCCTVKCEQCKQVVIKHFCA

SEQ ID NO: 2 - protein, 104 aa, Artificial Sequence
  Synthetic Peptide
AWSAPDYCLKNISLGLQGTEGRECLQSGKNLSQWERRSCKRLCTDCGLRVEEKKTEIISSCNCKFHWCCTVKCEQCKQVVIEHFCAGSSGGEQKLISEEDLLEI

SEQ ID NO: 3 - protein, 111 aa, Artificial Sequence
  Synthetic Peptide
AWSVNNFLEDSPDHCLKNISLGLQGTEGRECLQSGKNLSQWERRSCKRLCTDCGLRVEEKKTEIISSCNCKFHWCCTVKCEQCKQVVIKYFCAGSSGGEQKLISEEDLLEI

SEQ ID NO: 4 - protein, 173 aa, Artificial Sequence
  Synthetic Peptide
NGKAMQGVFEYYKSVTFVSNCGSHPSTTSKGSPINTQYVFKDNSSTIEGRYPYDVPDYALQASGGGGSVLEDLPDYCLKNISLGLQGTEGRECLQSGKNLSQWERRSCKRLCTDCGLHVEEKKIEIISSCNCKFHWCCTVKCEQCKQVVVKHFCAGSSGGEQKLISEEDLLEI

SEQ ID NO: 5 - protein, 114 aa, Artificial Sequence
  Synthetic Peptide
AWSVNNELIFLEDSPDYCLKNISLGLQGTEGRECLQSGKNLSQWERRSCKRLCTDCGLRVEERKTEIISSCNCKFHWCCTVKCEQCKQVVIKHFCAGSSGGEQKLISEEDLLEI

SEQ ID NO: 6 - protein, 120 aa, Homo sapiens
AIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 7 - protein, 120 aa, Homo sapiens
AVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 8 - protein, 108 aa, Homo sapiens
AAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 9 - protein, 108 aa, Homo sapiens
AAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 10 - protein, 115 aa, Homo sapiens
AVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 11 - protein, 115 aa, Homo sapiens
AVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 12 - protein, 110 aa, Homo sapiens
SIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 13 - protein, 118 aa, Homo sapiens
ASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 14 - protein, 109 aa, Homo sapiens
ASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 15 - protein, 121 aa, Homo sapiens
ATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 16 - protein, 108 aa, Homo sapiens
AIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 17 - protein, 114 aa, Homo sapiens
SEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 18 - protein, 114 aa, Homo sapiens
SEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 19 - protein, 111 aa, Homo sapiens
ATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 20 - protein, 111 aa, Homo sapiens
ATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 21 - protein, 116 aa, Homo sapiens
ALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 22 - protein, 113 aa, Homo sapiens
AVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 23 - protein, 137 aa, Homo sapiens
ALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 24 - protein, 143 aa, Xenopus laevis
ALKLEMDKRKMRSGNSADNRGAIADAFSSVAGSELIFLEDSPDYCLKNISLGLQGTEGRECLQSGKNLSQWERRSCKRLCTDCGLRVEEKKTEIISSCNCKFHWCCTVKCEQCKQVVIKHFCARRERDSNMLNTKRKNRGHRR

SEQ ID NO: 25 - protein, 137 aa, Homo sapiens
ALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 26 - protein, 225 aa, Homo sapiens
ANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDG

SEQ ID NO: 27 - protein, 215 aa, Homo sapiens
SWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNG

SEQ ID NO: 28 - protein, 213 aa, Homo sapiens
SWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDG

SEQ ID NO: 29 - protein, 220 aa, Homo sapiens
GYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSA

SEQ ID NO: 30 - protein, 220 aa, Homo sapiens
SYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSA

SEQ ID NO: 31 - protein, 219 aa, Homo sapiens
ASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDG

SEQ ID NO: 32 - protein, 235 aa, Homo sapiens
QVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDS

SEQ ID NO: 33 - protein, 263 aa, Homo sapiens
QLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVY

SEQ ID NO: 34 - protein, 232 aa, Homo sapiens
LWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHG

SEQ ID NO: 35 - protein, 213 aa, Homo sapiens
RIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNE

SEQ ID NO: 36 - protein, 210 aa, Homo sapiens
ALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNA

SEQ ID NO: 37 - protein, 192 aa, Homo sapiens
WSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQ

SEQ ID NO: 38 - protein, 191 aa, Homo sapiens
SVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHA

SEQ ID NO: 39 - protein, 220 aa, Homo sapiens
YFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYET

SEQ ID NO: 40 - protein, 223 aa, Homo sapiens
ASYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDS

SEQ ID NO: 41 - protein, 260 aa, Homo sapiens
PRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHR

SEQ ID NO: 42 - protein, 253 aa, Homo sapiens
NEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGR

SEQ ID NO: 43 - protein, 219 aa, Homo sapiens
IKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLS

SEQ ID NO: 44 - protein, 233 aa, Homo sapiens
NWMWLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKD

SEQ ID NO: 45 - protein, 213 aa, Xenopus laevis
WSVNNFLMTGPKAYLTYSASVAVGAQNGIEECKYQFAWERWNCPESTLQLATHNGLRSATRETSFVHAISSAGVMYTLTRNCSMGDFDNCGCDDSRNGRIGGRGWVWGGCSDNAEFGERISKLFVDGLETGQDARALMNLHNNEAGRLAVKETMKRTCKCHGISGSCSIQTCWLQLAEFRDIGNHLKIKHDQALKLEMDKRKMRSGNSADNRG

SEQ ID NO: 46 - protein, 290 aa, Drosophila sp.
QVEGKQKSGRGRGSMWWGIAKVGEPNNITPIMYMDPAIHSTLRRKQRRLVRDNPGVLGALVKGANLAISECQHQFRNRRWNCSTRNFSRGKNLFGKIVDRGCRETSFIYAITSAAVTHSIARACSEGTIESCTCDYSHQSRSPQANHQAGSVAGVRDWEWGGCSDNIGFGFKFSREFVDTGERGRNLREKMNLHNNEAGRAHVQAEMRQECKCHGMSGSCTVKTCWMRLANFRVIGDNLKARFDGATRVQVTNSLRATNALAPVSPNAAGSNSVGSNGLIIPQSGLVYGE

(19) United States
(12) Patent Application Publication
(10) Pub. No.: US 2014/0200179 A1
(43) Pub. Date: Jul. 17, 2014

(54) WNT COMPOSITIONS AND METHODS OF USE THEREOF

(71) Applicant: THE BOARD OF THE TRUSTEES OF THE LELAND STANFORD JUNIOR UNIVERSITY, Palo Alto, CA (US)

(72) Inventors: Kenan Christopher Garcia, Stanford, CA (US); Aron Levin, Basking Ridge, NJ (US)

(73) Assignee: THE BOARD OF THE TRUSTEES OF THE LELAND STANFORD JUNIOR UNIVERSITY, Palo Alto, CA (US)

(21) Appl. No.: 13/982,184

(22) PCT Filed: Jan. 26, 2012
(86) PCT No.: PCT/US12/22761

§ 371 Date: Mar. 14, 2014

Related U.S. Application Data
(60) Provisional application No. 61/462,130, filed on Jan. 28, 2011.

(51) Int. Cl. C07K 14/575

(52) U.S. Cl. CPC C07K 14/575
USPC 514/7.6; 530/399; 435/375; 435/7.92

(57) ABSTRACT

Wnt compositions and methods for their use are provided. Compositions of the invention comprise fragments of wnt polypeptides having a desired biological activity, which fragments are referred to herein as “mini-wnts”. These compositions and methods find particular use in determining bind to Wnt receptors; inhibiting Wnt signaling in a cell that expresses a Wnt receptor; in delivering a functional moiety to a cell that expresses a Wnt receptor; and as an immunogen for producing Wnt-specific antibodies.

### BACKGROUND OF THE INVENTION

Wnt proteins form a family of highly conserved secreted signaling molecules that regulate cell-to-cell interactions during embryogenesis. Wnt genes and Wnt signaling are also implicated in cancer. Wnt glycoproteins are thought to function as paracrine or autocrine signals active in several primitive cell types.

The Wnt growth factor family includes more than 19 genes identified in the mouse and in humans. The Wnt-1 proto-oncogene (int-1) was originally identified from mammary tumors induced by mouse mammary tumor virus (MMTV) due to an insertion of viral DNA sequence (Nusse and Varmus (1982) Cell 31:99-109). Expression of Wnt proteins varies, but is often associated with developmental process, for example in embryonic and fetal tissues. Wnts may play a role in local cell signaling. Biochemical studies have shown that much of the secreted Wnt protein can be found associated with the cell surface or extracellular matrix rather than freely diffusible in the medium.

Insights into the mechanisms of Wnt action have emerged from several systems: genetics in Drosophila and Caenorhabditis elegans; biochemistry in cell culture and ectopic gene expression in Xenopus embryos. Many Wnt genes in the mouse have been mutated, leading to very specific developmental defects. As currently understood, Wnt proteins bind to receptors of the Frizzled family, receptors of the ROR family, and the LRP5 receptor, LRP6 receptor, and FRL1 receptor on the cell surface. In the canonical Wnt/β-catenin signaling pathway, Wnt binding to Frizzled receptors results in nuclear localization of β-catenin, which then complexes with TCF to activate transcription of Wnt target genes.

Studies of mutations in Wnt genes have indicated a role for Wnts in growth control and tissue patterning. In Drosophila, wingless (wg) encodes a Wnt gene and wg mutations alter the pattern of embryonic ectoderm, neurogenesis, and imaginal disc outgrowth. In Caenorhabditis elegans, lin-44 encodes a Wnt, which is required for asymmetric cell divisions. Knock-out mutations in mice have shown Wnts to be essential for brain development, and the outgrowth of embryonic primordia for kidney, tail bud, and limb bud. Overexpression of Wnts in the mammary gland can result in mammary hyperplasia, and precocious alveolar development. Thus, Wnt signaling is involved in numerous events in animal development, including the proliferation of stem cells and the specification of the neural crest. Wnt proteins are therefore potentially important reagents in expanding specific cell types, and in treatment of conditions in vivo.

Data has indicated that in its native form, biologically active Wnt requires that the protein be palmitoylated on a conserved cysteine, see, inter alia, Willert et al. (2003) Nature 423:448-452; and Nusse (2005) Cell Research 15:28-32. These lipid groups lower the water solubility of the Wnt protein. Thus, to date, methods for isolation of biologically effective concentrations of active native and recombinant Wnt have required the use of detergents or liposomes (see Willert et al., supra, Zhao et al. (2009) Methods Enzymol. 465:331-347; and Morrell et al. (2008) PLoS One 3(8):e2930. The requirement for these chemical agents for the solubility of Wnt limits its pharmaceutical utility due to the problems of injecting these chemicals into animals. The development of pharmaceutically active wnt compositions that are water soluble is therefore of great interest.

### Publications

The biological activity of soluble wingless protein is described in van Leeuwen et al. (1994) Nature 24:368(6469):342-4. Biochemical characterization of Wnt-frizzled interactions using a soluble, biologically active vertebrate Wnt protein is described by Hsieh et al. (1999) Proc Natl Acad Sci USA 96(7):3546-51. Bradley et al. (1995) Mol Cell Biol 15(8):4616-22 describe a soluble form of wnt protein with mitogenic activity. Hoppler et al. (1996) Genes and Dev. 10:2805-2817 describe dominant negative Wnt polypeptides that are truncated Wnts (truncated Xwnt-8 and truncated mouse Wnt-1). Couso and Martinez-Arias (1994) Cell 79(2):259-72 describes a mutant allele of Dwnt-1 that encodes a secreted protein with a substantial carboxy-terminal deletion that has antimorphic effects. Other dominant negative Wnt polypeptides and methods for their use are described in WO2010/078458.

Willert et al. (2003) Nature 423:448-452 describes methods of purifying Wnt proteins with the aid of detergents. Morrell et al. (2008) PLoS One 3(8):e2930 describes the formulation of Wnt proteins into liposomes, and how Wnts packaged in liposomes retain biological activity in vivo. Patent publications include U.S. Pat. Nos. 7,335,643; and 7,153,832; and published U.S. Patent Application 20080226707.

### SUMMARY OF THE INVENTION

Wnt compositions and methods for their use are provided. Compositions of the invention comprise fragments of wnt polypeptides having a desired biological activity, which fragments are referred to herein as “mini-wnts”. Mini-wnt polypeptides of interest include fragments of native wnt proteins and derivatives thereof, e.g. analogs comprising one or more amino acid changes relative to the native sequence that enhance a property of interest, such as solubility, affinity or specificity for a targeted receptor. The fragments, particularly those of the Wnt C-terminus, may be water soluble. Compositions of interest include, without limitation, an effective dose of a mini-wnt polypeptide in a pharmaceutically acceptable excipient. Compositions may comprise additional agents, e.g. adjuvants and the like. Mini-wnt polypeptides may be produced synthetically; by various suitable recombinant methods, and the like, as known in the art.

These compositions and methods find particular use in inhibiting Wnt signaling in a cell that expresses a Wnt receptor, e.g. to inhibit aberrant cell proliferation; in delivering a functional moiety, e.g. a therapeutic or an imaging moiety, to a cell that expresses a Wnt receptor; and as an immunogen for producing Wnt-specific antibodies. Usually mini-wnts bind to one but not both of a Wnt co-receptor pair.

In some embodiments, the mini-wnt is a C-terminal, or “Cterm” mini-wnt, where a Cterm mini-wnt is a polypeptide that comprises or consists of the carboxy terminal domain of a wnt polypeptide, and that does not include the amino acid residues of the amino terminal domain. The delineation of the carboxy terminal domain is exemplified herein with multiple human Wnt proteins, e.g. as shown in FIG. 6. Wnt carboxy terminal domains may also be empirically identified by alignment with sequences provided herein. In one example, a Cterm mini-wnt amino acid sequence aligns by conserved residues with positions 298-370 of human Wnt1 and lacks the amino acid sequence that aligns with residues 1-257 of human Wnt1. Cterm min-wnts are generally water soluble.

In other embodiments, the mini-wnt is an N-terminal, or “Nterm”, mini-wnt, where an Nterm mini-wnt is a polypeptide that comprises or consists of the amino terminal domain of a wnt polypeptide, and that does not include the amino acid residues of the carboxy terminal domain. The delineation of the amino terminal domain is exemplified herein with multiple human Wnt proteins, e.g. as shown in FIG. 8. Wnt amino terminal domains may also be empirically identified by alignment with sequences provided herein. In one example, an Nterm mini-wnt amino acid sequence aligns by conserved residues with positions 34-247 of human Wnt1 and lacks the amino acid sequence that aligns with residues corresponding to residues 288-370 of human Wnt1. Nterm mini-wnts may be water soluble or may be lipid soluble.

In some embodiments, the mini-wnt polypeptide is fused or conjugated to a functional moiety, which may be a therapeutic moiety, e.g. a cytotoxic moiety, or a moiety that targets a cell for ADCC- or CDC-directed cell death. Therapeutic moieties of interest include those that alters the cell's activity. In some embodiments, the functional moiety is an imaging moiety, e.g. a fluorophore, luminophore, radioisotope, etc. In some embodiments the functional moiety is an oligomerizing moiety that induces oligomerization of the mini-wnt into homo-dimers, -trimers, -tetramers or more, or hetero-dimers, -trimers, -tetramers or more by, e.g. using zippers or Fc polypeptides, or other avidity-enhancing chemical or protein agents. In some embodiments the functional moiety is a protein or chemical moiety that extends the half-life and/or increases the size of the mini-wnt polypeptide.

In some aspects of the invention, a method is provided for delivering a functional moiety to a cell, comprising contacting a cell of interest expressing a Wnt receptor with a mini-wnt comprising a functional moiety of interest. In other embodiments, methods are provided for inhibiting Wnt signaling in a cell. In such methods, a cell expressing a Wnt receptor is contacted with a concentration of a mini-wnt polypeptide that is effective to inhibit signaling, e.g. to reduce signaling by 25%, 50%, 75%, 90%, 95%, or more, relative to the signaling in the absence of the mini-wnt. Such signaling inhibition may inhibit proliferation of the targeted cell, or may otherwise interfere with Wnt-signaling pathways active in the targeted cell.

In the methods of the invention, where the Wnt receptor is a Fz protein, a ROR protein, or an Ryk protein, the mini-wnt polypeptide is usually a Cterm mini-wnt. Where the Wnt receptor is LRP5, LRP6, or FRL1/crypto, usually the mini-wnt polypeptide is an Nterm mini-wnt. In some methods, the receptor-expressing cell is contacted in vitro. In other embodiments, the receptor-expressing cell is contacted in vivo. Cells of interest include a wide variety of Wnt-receptor expressing cells, as are known in the art.

Methods are provided for making Wnt-specific antibodies, the method comprising immunizing an animal with an effective dose of a Cterm or Nterm mini-wnt polypeptide, optionally formulated with an adjuvant. Polyclonal antibodies may be obtained from serum, or cells from the animal may be used in the production of monoclonal antibodies, as a source of polynucleotides for the generation of recombinantly produced antibodies, etc.

Methods are provided for determining the cognate receptor for a Wnt protein, the method comprising contacting a candidate Wnt receptor or fragment thereof with a mini-wnt polypeptide that corresponds to a Wnt protein of interest; and determining the binding of the mini-wnt to the candidate receptor, wherein the presence of specific binding is indicative that the Wnt protein is a ligand for the candidate receptor. Receptors of interest include Fz protein, a ROR protein, or an Ryk protein, which may be contacted with a Cterm mini-wnt; and LRP5, LRP6, or FRL1/crypto, which may be contacted with an Nterm mini-wnt. Various binding assays find use, for example utilizing cells expressing the candidate receptor, but of particular interest are assays that can be performed in solution, e.g. utilizing a soluble fragment of the receptor for binding, including without limitation interactions between Fz-CRD polypeptides and water soluble Cterm mini-wnts polypeptides.

A benefit of the compositions and methods of the invention is the specificity of targeting, where the mini-wnt targets the same cells as the native wnt protein from which it is derived. For example, mini-wnts selectively inhibit Wnt signaling in those cells that are responsive to the Wnt parent protein. Inhibitors of Wnt signaling known in the art, e.g. antibodies that bind to specific Wnt receptors, will not bind to all Wnt receptors targeted by the Wnt parent, but rather only those Wnt receptor(s) for which they have specificity. In addition, a benefit of the water soluble forms of mini-wnts is the lack of a requirement for formulation additives that might limit their therapeutic utility.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Evolution of a soluble Xenopus Wnt8 (“Xwnt8”). Panel A (left side) depicts a cartoon of a Wnt molecule expressed on the surface if yeast. The Wnt molecule harbors several mutations (stars) that result in water solubility of the molecule. Connected to the C-terminal end of the Wnt is a “Myc” tag that is used in the experiments to detect the expressed Wnt using an antibody to Myc. Also in panel A (right side) is a cartoon schematic of the Wnt-binding domain of the Wnt receptor Frizzled, in this case Fz5. This domain is called a “CRD.” The cartoon of the CRD shows 4 CRD molecules arrayed bound to a tetravalent streptavidin molecule (center cross cartoon) that is linked to a fluorescent dye for visualization. In Panel B, it is shown the Fz5 CRD contains biotin added to its C-terminal end so that it can bind to streptavidin. This is shown in an SDS-PAGE gel where the Fz5 CRD “shifts” up to bind to streptavidin in the gel. This “shift” shows that biotin is present on the Fz5 and Fz8 CRD. Panel C shows the sequence of full-length Xenopus Wnt8 subdivided into Nterm (purple shading) mini-Wnt, linker (green shaded), and C-term mini-Wnt (red shaded). The bock red arrows denoted B7, A12, B2, and A3 indicate different sequence boundaries of mini-wnts. The actual boundary for each mini-Wnt is variable (see FIG. 6), and can extend several residues into the linker.

FIG. 2. Validation of individual A clones. For each clone, anti-myc antibody binding to the Wnt C-terminal epitope shown in FIG. 1, and Fz5 and Fz8CRD binding to yeast displayed XWnt8 are shown in the top FACS panels. In the companion panels underneath linked by the brown block arrow, is shown that the anti-Myc and Fz5/Fz8-CRD binding is lost when the Wnt is cleaved off the yeast with a 3C protease. Since the yeast displayed Wnt contains a 3C protease cleavage site between the Wnt and the yeast, loss of binding to Myc and Fz-CRD indicates a specific interaction with the displayed Wnt, and not with the yeast in a non-specific way. In this way, the loss of binding upon cleavage is a very stringent test of biding specificity. (A) Validation of clone wnt A3. The construct AGA2-Linker-3C site-GS-AWSAPDYCLKNISLGLQGTEGRECLQSGKNLSQWERRSCKRLCTDCGLRVEEKKTEIISS CNCKFHWCCTVKCEQCKQVVIEHFCAGSSGGEQKLISEEDLLEI** was used. (B) Validation of clone wnt A12. The construct AGA2-Linker-3C site-GS-AWSVNNFLEDSPDHCLKNISLGLQGTEGRECLQSGKNLSQWERRSCKRLCTDCGLRVEE KKTEIISSCNCKFHWCCTVKCEQCKQVVIKYFCAGSSGGEQKLISEEDLLEI** was used.

FIG. 3. Validation of individual B clones. For each clone, anti-myc antibody binding to the Wnt C-terminal epitope shown in FIG. 1, and Fz5 and Fz8CRD binding to yeast displayed XWnt8 are shown in the top FACS panels. In the companion panels underneath linked by the brown block arrow, is shown that the anti-Myc and Fz5/Fz8-CRD binding is lost when the Wnt is cleaved off the yeast with a 3C protease. Since the yeast displayed Wnt contains a 3C protease cleavage site between the Wnt and the yeast, loss of binding to Myc and Fz-CRD indicates a specific interaction with the displayed Wnt, and not with the yeast in a non-specific way. In this way, the loss of binding upon cleavage is a very stringent test of binding specificity. (A) Validation of clone wnt B2. The construct AGA2-Linker-3C site-GSNGKAMQGVFEYYKSVTFVSNCGSHPSTTSKGSPINTQYVFKDNSSTIEGRYPYDVPDYAL QASGGGGSVLEDLPDYCLKNISLGLQGTEGRECLQSGKNLSQWERRSCKRLCTDCGLHV EEKKIEIISSCNCKFHWCCTVKCEQCKQVVVKHFCAGSSGGEQKLISEEDLLEI** was used (B) Validation of clone wnt B7. The construct AGA2-Linker-3C site-GSAWSVNNELIFLEDSPDYCLKNISLGLQGTEGRECLQSGKNLSQWERRSCKRLCTDCGLRV EERKTEIISSCNCKFHWCCTVKCEQCKQVVIKHFCAGSSGGEQKLISEEDLLEI** was used.

FIG. 4. Production of recombinant, water-soluble C-terminal mini-Wnt8 domain from baculovirus infected insect cells, and demonstration of the interaction with Fz5 CRD. In step 1, the B7 boundary version of mini-Xwnt8 was expressed from insect cells with a C-terminal hexahistidine tag (upper, far left cartoon). In step 2, this protein was then purified by a superdex-75 gel filtration chromatography (and accompanying SDS-GEL stained with coommassie blue) where it was shown to elute from the column at a position representing a properly folded protein of the correct MW as mini-Wnt. In step 3, this min-Wnt8 is then added to a Fz5-CRD-Fc fusion, and in step 4 re-analyzed over gel filtration. Fractionation of the mixture over a Superdex 75 column followed by electrophoresis on a non-reducing polyacrylamide gel demonstrated the presence of the Cterm mini-wnt8 and Fz5CRD-Fc fusion in the same fractions, with the Cterm mini-wnt8 eluting in earlier fractions than in the absence of Fz5CRD-Fc. In step 5,Fz immunoprecipitation of the Fz5CRD-Fc fusion with Protein A precipitated the Cterm mini-wnt8 protein as well. Thus formation of a specific binding interaction between mini-XWnt8 and Fz5-CRD was demonstrated in two ways: step 4-co-elution using gel filtration, and step 5-co-immunoprecipitation.

FIG. 5. Binding affinity measurements of mini-XWnt8 (amino acid residues 248-338) of mFz8/5-CRD by Surface Plasmon Resonance (Biacore). The Fz8-CRD or mFz5 CRD is tethered to a streptavidin-coated CM4 Biacore chip via a C-terminal biotin. Data demonstrates a direct interaction of purified, insect expressed mini-XWnt8 C-term fragment with both Fz5 CRD and Fz8 CRD. At the bottom of the figure, the raw curve fitting data and results are shown.

FIG. 6. Alignment of the C-terminal domain of human Wnts with C-terminal domain of Wnt8 from Xenopus laevis (NM_{—}001088168) and Wnt5 (“Drome”) from Drosophila melanogaster.

FIG. 7. Interaction between the LRP6 extracellular domain and the N-terminal mini-Wnt of XWnt8 and mutants thereof. (a) Interaction between wild-type Nterm mini-wnt of XWnt8 and LRP6. (b) Interaction between clones of Nterm mini-wnts of XWnt8 from error prone yeast library and LRP6. Clones were analyzed by FACS with anti-c-myc (histograms; c-myc antibody: 1:250 primary, 1:100 secondary) to verify surface display, and with LRP6(E1E2)-Fc (dot plots; LRP6(E1E2)-Fc: 2 μM, 1:25 secondary) to demonstrate that the domain binds to LRP6. Peaks in the gated domains of the histograms indicate positive signal for c-myc and hence the presence of Nterm mini-wnt-8. Populations in the lower right quadrant of the dot plots indicate positive signal for LRP6. Note that the clones in (b) all have comparable FACS plots to those in (a), which were generated from wild-type N-term mini-wnt. In some clones, a Cys55Ala or a Ser187Ala mutation was incorporated into the parent XWnt8 cDNA before making the library, to ensure that the polypeptide was not modified by the addition of palmitate lipid groups. These mutation sites were chosen based on published literature designating analogous positions of 55 and 187 in Wnt3A as the sites of palmitoylation (see, for example, Willert et al. (2003) supra.) A non-mutated wild-type staining of XWnt8 N-term domain is also provided, which also demonstrates substantial binding to Lrp6. Thus, these data demonstrate water-soluble Nterm mini-Wnt binding to LRP6.

FIG. 8. Alignment of the N-terminal domain of human Wnts with N-terminal domain of Xenopus laevis Wnt8 and Drosophila melanogasterWnt5 (“Drome”).

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Mini-wnt compositions and methods for their use are provided. These compositions and methods find particular use in inhibiting Wnt signaling in a cell that expresses a Wnt receptor, e.g. to inhibit aberrant cell proliferation; in delivering a therapeutic moiety to a cell that expresses a Wnt receptor; in delivering an imaging moiety to a cell that expresses a Wnt receptor; and in producing Wnt-specific antibodies. These and other objects, advantages, and features of the invention will become apparent to those persons skilled in the art upon reading the details of the compositions and methods as more fully described below.

A “Wnt protein” is a member of the family of highly conserved secreted signaling molecules that play key roles in both embryogenesis and mature tissues. The terms “Wnts” or “Wnt gene product” or “Wnt polypeptide” when used herein encompass native sequence Wnt polypeptides, Wnt polypeptide variants, Wnt polypeptide fragments and chimeric Wnt polypeptides. A “mini-wnt polypeptide” is a polypeptide that is a fragment of a full-length Wnt protein that retains the ability of the full-length Wnt protein from which it was derived to specifically bind to one or more Wnt receptors. The binding of the mini-wnt to a Wnt receptor may have a dominant negative effect, in that signaling from the Wnt receptor is inhibited.

The term “native sequence Wnt polypeptide” refers to Wnt polypeptides comprising sequence as they are found in nature. For example, human native sequence Wnt proteins of interest in the present application include the following: Wnt-1 (GenBank Accession No. NM_{—}005430); Wnt-2 (GenBank Accession No. NM_{—}003391); Wnt-2B (Wnt-13) (GenBank Accession No. NM_{—}004185 (isoform 1), NM_{—}024494.2 (isoform 2)), Wnt-3 (RefSeq.: NM_{—}030753), Wnt3a (GenBank Accession No. NM_{—}033131), Wnt-4 (GenBank Accession No. NM_{—}030761), Wnt-5A (GenBank Accession No. NM_{—}003392), Wnt-5B (GenBank Accession No. NM_{—}032642), Wnt-6 (GenBank Accession No. NM_{—}006522), Wnt-7A (GenBank Accession No. NM_{—}004625), Wnt-7B (GenBank Accession No. NM_{—}058238), Wnt-8A (GenBank Accession No. NM_{—}058244), Wnt-8B (GenBank Accession No. NM_{—}003393), Wnt-9A (Wnt-14) (GenBank Accession No. NM_{—}003395), Wnt-9B (Wnt-15) (GenBank Accession No. NM_{—}003396), Wnt-10A (GenBank Accession No. NM_{—}025216), Wnt-10B (GenBank Accession No. NM_{—}003394), Wnt-11 (GenBank Accession No. NM_{—}004626), Wnt-16 (GenBank Accession No. NM_{—}016087)). Although each member has varying degrees of sequence identity with the family, all encode small (i.e., 39-46 kD), acylated, palmitoylated, secreted glycoproteins that contain 23-24 conserved cysteine residues whose spacing is highly conserved (McMahon, A P et al., Trends Genet. 1992; 8: 236-242; Miller, J R. Genome Biol. 2002; 3(1): 3001.1-3001.15). Other native sequence Wnt polypeptides of interest in the present invention include orthologs of the above from any mammal, including domestic and farm animals, and zoo, laboratory or pet animals, such as dogs, cats, cattle, horses, sheep, pigs, goats, rabbits, rats, mice, frogs, zebra fish, fruit fly, worm, etc.

A “native sequence” mini-wnt polypeptide is a fragment of the amino acid sequence of a sequence Wnt polypeptide. Such native sequence polypeptides can be isolated from cells producing endogenous Wnt protein or can be produced by recombinant or synthetic means. Thus, a native sequence mini-wnt polypeptide can have the amino acid sequence comprised by, e.g. naturally occurring human Wnt polypeptide, murine Wnt polypeptide, or polypeptide from any other mammalian species, or from non-mammalian species, e.g. Drosophila, C. elegans, and the like.

A “variant” mini-wnt polypeptide means a biologically active polypeptide as defined below having less than 100% sequence identity with a native Wnt sequence over the length of the fragment. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, biologically active variants will have an amino acid sequence having at least about 75% sequence identity, about 80% sequence identity, about 85% amino acid sequence identity, about 90% amino acid sequence identity with a native sequence polypeptide, preferably at least about 95%, more preferably at least about 99% sequence identity. Various methods known in the art may be utilized in developing such variant polypeptides.

A “chimeric” mini-wnt polypeptide is a polypeptide comprising a mini-wnt polypeptide fused or conjugated to a heterologous polypeptide. The chimeric mini-wnt polypeptide will generally share at least one biological property with the initial mini-wnt polypeptide. Examples of chimeric polypeptides include mini-wnt polypeptides fused to one or more functional moieties such as a therapeutic moiety, an imaging moiety, an epitope tag. Typically, when the functional moiety is a polypeptide moiety, the moiety has sufficient residues to provide a function, e.g. promoting multimerization, promoting cell death, altering cell function, fluorescence signal, an epitopic sequence, etc., yet is short enough such that it does not interfere with biological activity of the mini-wnt polypeptide. Suitable tag polypeptides for use as an epitope generally have at least six amino acid residues and usually between about 6-250 amino acid residues.

By “water soluble” it is meant a composition that is soluble in aqueous buffers in the absence of detergent, usually soluble at a concentration that provides a biologically effective dose of the polypeptide. Compositions that are water soluble form a substantially homogenous composition that has a specific activity that is at least about 5% that of the starting material from which it was purified, usually at least about 10%, 20%, or 30% that of the starting material, more usually about 40%, 50%, or 60% that of the starting material, and may be about 50%, about 90% or greater. Mini-wnt compositions of the present invention typically form a substantially homogeneous solution at concentrations of at least 25 μM and higher, e.g. at least 25 μM, 40 μM, or 50 μM, usually at least 60 μM, 70 μM, 80 μM, or 90 μM, sometimes as much as 100 μM, 120 μM, or 150 W. In other words, mini-wnt compositions of the present invention typically form a substantially homogeneous solution at concentrations of about 0.1 mg/ml, about 0.5 mg/ml, of about 1 mg/ml or more.

“Wnt protein signaling” or “Wnt signaling” is used herein to refer to the mechanism by which a biologically active Wnt exerts its effects upon a cell to modulate a cell's activity. Wnt proteins modulate cell activity by binding to Wnt receptors, including proteins from the Frizzled (Fz) family of proteins, proteins from the ROR family of proteins, the proteins LRP5, LRP6 from the LRP family of proteins, the protein FRL1/crypto, and the protein Derailed/Ryk. Once activated by Wnt binding, the Wnt receptor(s) will activate one or more intracellular signaling cascades. These include the canonical Wnt signaling pathway; the Wnt/planar cell polarity (Wnt/PCP) pathway; the Wnt-calcium (Wnt/Ca²⁺) pathway (Giles, R H et al. (2003) Biochim Biophys Acta 1653, 1-24; Peifer, M. et al. (1994) Development 120: 369-380; Papkoff, J. et al (1996) Mol. Cell. Biol. 16: 2128-2134; Veeman, M. T. et al. (2003) Dev. Cell 5: 367-377); and other Wnt signaling pathways as is well known in the art. For example, activation of the canonical Wnt signaling pathway results in the inhibition of phosphorylation of the intracellular protein β-catenin, leading to an accumulation of β-catenin in the cytosol and its subsequent translocation to the nucleus where it interacts with transcription factors, e.g. TCF/LEF, to activate target genes. Activation of the Wnt/PCP pathway activates RhoA, c-Jun N-terminal kinase (JNK), and nemo-like kinase (NLK) signaling cascades to control such biological processes as tissue polarity and cell movement. Activation of the Wnt/Ca2+ by, for example, binding of Wnt-4, Wnt-5A or Wnt-11, elicits an intracellular release of calcium ions, which activates calcium sensitive enzymes like protein kinase C(PKC), calcium-calmodulin dependent kinase II (CamKII) or calcineurin (CaCN). By assaying for activity of the above signaling pathways, the biological activity of a Wnt composition can be readily determined. A “biologically active mini-wnt” is a mini-wnt composition that is able to specifically bind to Wnt receptor and modulate Wnt signaling when provided to a cell in vitro or in vivo, that is, when administered to an animal, e.g. a mammal. Frequently mini-wnt polypeptides are dominant negative, or competitive, inhibitors of wnt signaling.

The term “specific binding” refers to that binding which occurs between such paired species as enzyme/substrate, receptor/ligand, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or ligand/receptor interaction. One may determine the biological activity of a mini-wnt protein in a composition by determining the level of activity in a functional assay after in vivo administration, e.g. decelerating bone regeneration, downregulation of stem cell proliferation, etc., quantitating the amount of mini-wnt protein present in a non-functional assay, e.g. immunostaining, ELISA, quantitation on coomassie or silver stained gel, etc., and determining the ratio of in vivo biologically active mini-wnt to total mini-wnt. A mini-wnt composition that is biologically active is one that modulates the activity of a Wnt protein by at least about 40%, about 60%, more usually by about 70%, 75%, or 80%, often by about 85%, 90%, or 95%, sometimes by as much as 100%, i.e. complete abrogation of Wnt signaling.

The terms “Wnt antagonist”, “Wnt inhibitor”, and “inhibitor of Wnt signaling” are used interchangeably herein to mean an agent that antagonizes, inhibits, or negatively regulates Wnt modulation of a cell's activity. Likewise, the phrases “antagonizing Wnt signaling” and “inhibiting Wnt signaling” are used interchangeably herein to mean antagonizing, inhibiting, or otherwise negatively regulating Wnt modulation of a cell's activity.

“Fz”, “Fz proteins” and “Fz receptors” is used herein to refer to proteins of the Frizzled receptor family. These proteins are seven-pass transmembrane proteins (Ingham, P. W. (1996) Trends Genet. 12: 382-384; Yang-Snyder, J. et al. (1996) Curr. Biol. 6: 1302-1306; Bhanot, P. et al. (1996) Nature 382: 225-230) that comprise a CRD domain. There are ten known members of the Fz family (Fz1 through Fz10), any of which may server as receptors of Wnts. Fz receptors mediate a number of Wnt biological activities, including but not limited to the modulation of synapse formation,

“LRP”, “LRP proteins” and “LRP receptors” is used herein to refer to proteins of the low density lipoprotein receptor-related protein family. These receptors are single-pass transmembrane proteins that bind and internalize ligands in the process of receptor-mediated endocytosis. LRP proteins LRP5 (GenBank Accession No. NM_{—}002335.2) and LRP6 (Gen Bank Accession No. NM_{—}002336.2) are included in the Wnt receptor complex.

Ryk (the Drosophila homolog of which is Derailed) is an atypical member of the family of growth factor receptor protein tyrosine kinases, differing from other members at a number of conserved residues in the activation and nucleotide binding domains. The protein sequences for Ryk may be found at GenBank Accession No. NM_{—}001005861 (isoform 1) and NM_{—}002958.3 (isoform 2). Ryk functions as a receptor for Wnt proteins, usually as a coreceptor of Fzs, and mediates Wnt biological activities such as the modulation of osteoblast differentiation, cell migration, cell-fate determination, axon guidance, and neurite outgrowth, including motoneuron target selection and synaptogenesis at the neuromuscular junction. Activation of the Wnt/Ryk pathway by injury inhibits axon regeneration.

“RORs”, “ROR proteins” and “ROR” receptors” is used herein to refer to the ROR1 and ROR2 proteins of the Receptor Tyrosine Kinase-Like Orphan Receptor family. The protein sequence for ROR1 may be found at GenBank Accession No. NM_{—}005012.2 (isoform 1) and NM_{—}001083592.1 (isoform 2). The protein sequence for ROR2 may be found at GenBank Accession No. NM_{—}004560.3. ROR1 and ROR2 function as receptors of Wnt proteins, usually as coreceptors of Fzs.

“EGF-CFC proteins” and “EGF-CFC receptors” is used herein to refer to proteins encoded by the epidermal growth factor (EGF)— crypto, FRL-1, cryptic family. These proteins include Cripto (also called “CR-1” and “Tdgf1”, for teratocarcinoma-derived growth factor 1, GenBank Accession No. NM_{—}003212.3 (isoform 1) and NM_{—}001174136.1 (isoform 2)) and Cryptic (also called “CFC1”, GenBank Accession No. NM_{—}032545.2). EGF-CFC proteins share a variant EGF-like motif, a conserved cysteine-rich domain, and a C-terminal hydrophobic region. These proteins are Wnt receptors, and play key roles in intercellular signaling pathways during vertebrate embryogenesis and tumor growth.

By “comprising” it is meant that the recited elements are required in the composition/method/kit, but other elements may be included to form the composition/method/kit etc. within the scope of the claim. For example, a composition comprising a mini-wnt polypeptide is a composition that may comprise other elements in addition to mini-wnt polypeptide(s), e.g. functional moieties such as polypeptides, small molecules, or nucleic acids bound, e.g. covalently bound, to the mini-wnt polypeptide; agents that promote the stability of the mini-wnt composition, agents that promote the solubility of the mini-wnt composition, adjuvants, etc. as will be readily understood in the art, with the exception of elements that are encompassed by any negative provisos. As another example, a mini-wnt polypeptide that comprises Wnt amino acid sequence corresponding to, e.g. residues 298-370 of human Wnt or, e.g. residues 34-247, may comprise Wnt amino acid sequence in addition to that sequence with the exception of any sequence recited by negative provisos.

By “consisting essentially of”, it is meant a limitation of the scope of composition or method described to the specified materials or steps that do not materially affect the basic and novel characteristic(s) of the subject invention. For example, a mini-wnt polypeptide “consisting essentially of” a disclosed sequence has the amino acid sequence of the disclosed sequence plus or minus about 5 amino acid residues at the boundaries of the sequence based upon the full length parent Wnt sequence from which it was derived, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

By “consisting of”, it is meant the exclusion from the composition, method, or kit of any element, step, or ingredient not specified in the claim. For example, a mini-wnt polypeptide “consisting of” a disclosed sequence consists only of the disclosed amino acid sequence.

By “functional moiety” or “FM” it is meant a polypeptide, small molecule or nucleic acid composition that confers a functional activity upon a composition. Examples of functional moieties include, without limitation, therapeutic moieties, binding moieties, and imaging moieties.

By “therapeutic moiety”, or “TM”, it is meant a polypeptide, small molecule or nucleic acid composition that confers a therapeutic activity upon a composition. Examples of therapeutic moieties include cytotoxins, e.g. small molecule compounds, protein toxins, and radiosensitizing moieties, i.e. radionuclides etc. that are intrinsically detrimental to a cell; agents that alter the activity of a cell, e.g. small molecules, peptide mimetics, cytokines, chemokines; and moieties that target a cell for ADCC or CDC-dependent death, e.g. the Fc component of immunoglobulin.

By an “imaging moiety”, or “IM”, it is meant a non-cytotoxic agent that can be used to locate and, optionally, visualize cells, e.g. cells that have been targeted by compositions of the subject application.

An oligomerizing moiety is a polypeptide, small molecule or nucleic acid composition that induces oligomerization of the mini-wnt into homo-dimers, -trimers, -tetramers or more, or hetero-dimers, -trimers, -tetramers or more by, e.g. using zippers, or Fc polypeptides, biotin and avidin/streptavidin, or other avidity-enhancing chemical or protein agents as known in the art.

The phrases “Wnt-mediated condition” and “Wnt-mediated disorder” are used interchangeably herein to describe a condition, disorder, or disease state characterized by aberrant or undesirable Wnt signaling. In a specific aspect, the aberrant Wnt signaling is a level of Wnt signaling in a cell or tissue suspected of being diseased that exceeds the level of Wnt signaling in a similar non-diseased cell or tissue. Examples of Wnt-mediated disorders include those associated with aberrant angiogenesis, e.g. retinopathies, and those associated with aberrant proliferation, e.g. cancer.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Compositions

Mini-wnt compositions and methods for their use are provided. Mini-wnt compositions are compositions that comprise a mini-wnt polypeptide. As discussed above, a mini-wnt polypeptide is a polypeptide that is a fragment of a full-length Wnt protein that retains the ability of the full-length Wnt protein from which it was derived to specifically bind to at least one Wnt receptor. Unlike a full-length Wnt protein, which may simultaneously bind to two distinct co-receptors, a mini-wnt typically binds to only one of the co-receptors. A mini-wnt polypeptide is usually at least about 40 amino acids in length, usually at least about 50 amino acids in length, and not more than about 120 amino acids in length, usually not more than about 100 amino acids in length, and in some embodiments be from about 80 to about 100 amino acids in length.

In some embodiments, the mini-wnt polypeptide is a C-terminal mini-wnt polypeptide, also referred to herein as “C-terminal mini-wnt” or “Cterm mini-wnt”. A Cterm mini-wnt is a polypeptide composition derived from, i.e. comprising of sequence from, the C terminal domain of a Wnt protein that binds to a Fz, ROR, and/or Ryk Wnt receptor. In some embodiments, the Cterm mini-wnt is water soluble.

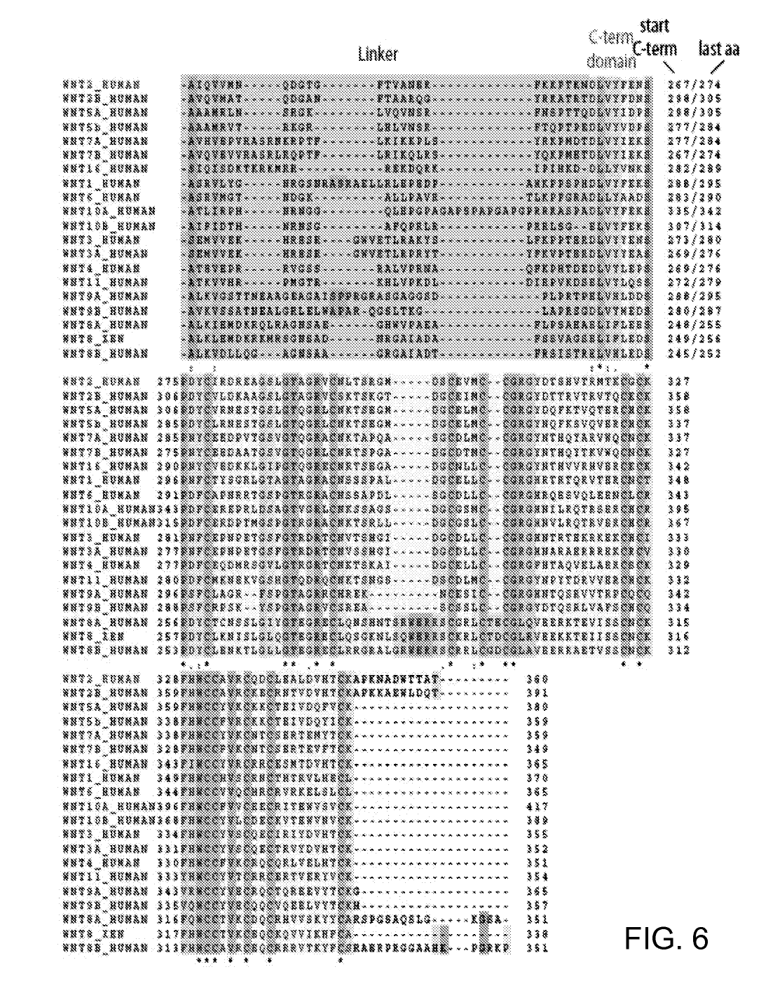

A Cterm mini-wnt comprises or consists of the carboxy terminal domain of a wnt polypeptide, and does not include the amino acid residues of the amino terminal domain. The delineation of the carboxy terminal domain is exemplified herein with multiple human Wnt proteins, e.g. as shown in FIG. 6, and may consist of substantially all the amino acid residues set forth in the delineated domain, or may be truncated by 1, 2, 3, 4, 5, 6, 7, 8, 9, or more amino acid residues at either the amino or carboxy terminus of the delineated domain. Cterm mini-wnts may also be empirically identified by alignment with sequences provided herein. In one example, a Cterm mini-wnt amino acid sequence aligns by conserved residues with positions 298-370 of human Wnt1 and lacks the amino acid sequence that aligns with residues 1-257 of human Wnt1 or may be truncated as indicated above. In some embodiments a Cterm mini-wnt is a variant or analog as defined above. In some embodiments, the variation, or mutation, alters the affinity for its cognate receptor; solubility; and/or specificity for its cognate receptor.

In some embodiments, the mini-wnt polypeptide is an N-terminal mini-wnt polypeptide, also referred to herein as “N-terminal mini-wnt” or “Nterm mini-wnt”. An Nterm mini-wnt is a polypeptide composition derived, i.e. consisting of sequence from, an N terminal domain of a Wnt protein that binds to LRP5, LRP6, and/or crypto. In some embodiments, the Nterm mini-wnt is water-soluble, in other embodiments the Nterm mini-wnt is lipid soluble.

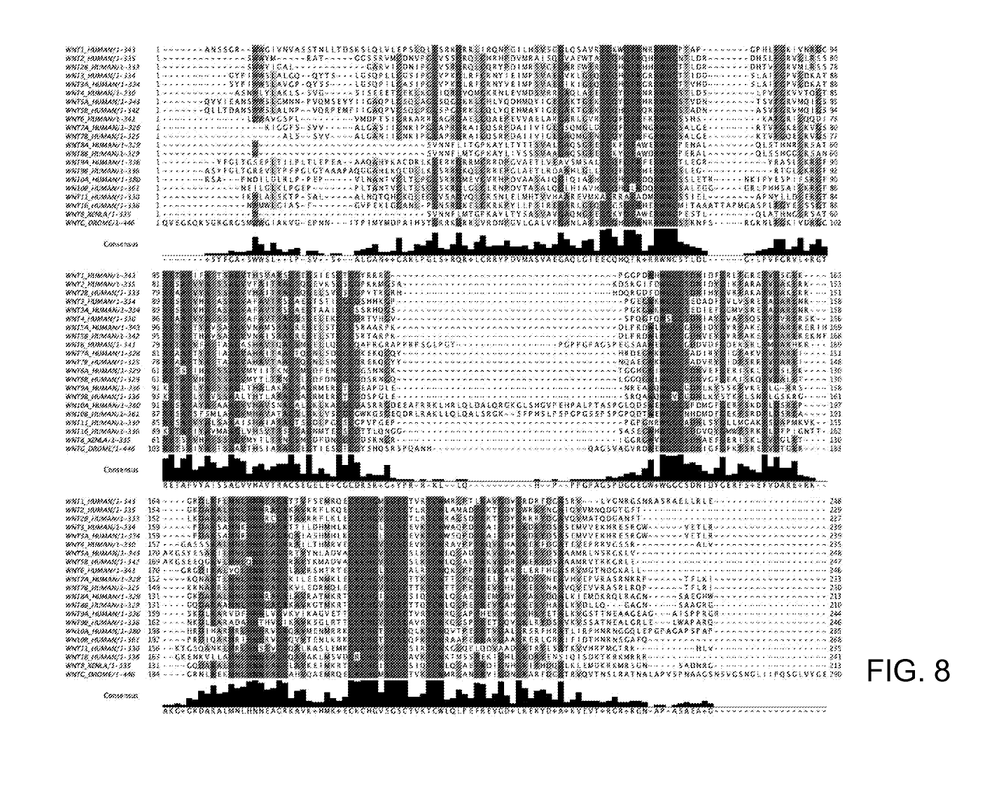

In some embodiments, an N-terminal mini-wnt polypeptide is a polypeptide that comprises or consists of the amino terminal domain of a wnt polypeptide, and does not include the amino acid residues of the carboxy terminal domain. The delineation of the amino terminal domain is exemplified herein with multiple human Wnt proteins, e.g. as shown in FIG. 8, and may consist of substantially all the amino acid residues set forth in the delineated domain, or may be truncated by 1, 2, 3, 4, 5, 6, 7, 8, 9, 10 or more amino acid residues at either the amino or carboxy terminus of the delineated domain. Nterm mini-wnts may also be empirically identified by alignment with sequences provided herein. In one example, an Nterm mini-wnt amino acid sequence aligns by conserved residues with positions 34-247 of human Wnt1 and lacks the amino acid sequence that aligns with residues corresponding to residues 288-370 of human Wnt1 or may be truncated as indicated above. Nterm mini-wnts may be water soluble or may be lipid soluble. In some embodiments a Cterm mini-wnt is a variant or analog as defined above. In some embodiments, the variation, or mutation, alters the affinity for its cognate receptor; solubility; and/or specificity for its cognate receptor. In some such embodiments, an amino acid substitution is made at the residue that is aligned to residues 93 and/or 224 of human Wnt 1, such that the residue corresponding to residues Cys⁵⁵ and/or Ser¹⁸⁷ of human Wnt 1 is now an alanine.

Cterm mini-wnt polypeptides and Nterm mini-wnt polypeptides that correspond to Wnt proteins from any species of organism, e.g. mouse, rat, cat, chicken, fruit fly, frog, zebra fish, dog, worm, etc., find use in the subject compositions. The polypeptide sequence for such mini-wnts may readily be determined by performing an alignment of the homolog or ortholog of interest with the provided Wnt sequences shown in FIGS. 6 and 8, using alignment software such as NCBI BLAST, ClustalW, or other software as is well known in the art. Compositions comprising a mini-wnt polypeptide may comprise various elements, with the exception of those elements that are specifically set forth herein as excluded from the recited mini-wnt polypeptide, for example the mini-wnt may be fused to an exogenous polypeptide. The site at which the fusion is made may be selected in order to optimize the biological activity, secretion or binding characteristics of the polypeptide. The optimal site will be determined by routine experimentation.

For example, compositions comprising a mini-wnt polypeptide optionally include polypeptides fused to the mini-wnt polypeptide to further increase their solubility. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

As another example, compositions comprising a mini-wnt polypeptide may optionally include modifications to the mini-wnt polypeptide to improve stability. For example, the peptide may be PEGylated, where the polyethyleneoxy group provides for enhanced lifetime in the blood stream. The polypeptide may be fused to another polypeptide to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

Stable plasma proteins are proteins which typically exhibit in their native environment an extended half-life in the circulation, i.e. greater than about 20 hours. Examples of suitable stable plasma proteins are immunoglobulins, albumin, lipoproteins, apolipoproteins and transferrin. The polypeptide agent typically is fused to the plasma protein, e.g. IgG at the N-terminus of the plasma protein or fragment thereof which is capable of conferring an extended half-life upon the polypeptide. Increases of greater than about 100% on the plasma half-life of the polypeptide are satisfactory. Ordinarily, the polypeptide is fused C-terminally to the N-terminus of the constant region of immunoglobulins in place of the variable region(s) thereof, however N-terminal fusions may also find use. Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods.

In some embodiments, the mini-wnt is modified without altering its sequence. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Mini-wnt polypeptides for use in the subject compositions and methods may be modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The mini-wnt polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.\

Alternatively, the mini-wnt polypeptide may be prepared by recombinant DNA technology in a cellular or cell-free polypeptide synthesis system, using any one of the many systems known in the art. Construction of suitable vectors containing one or more of the above-listed components employs standard ligation techniques. Isolated plasmids or DNA fragments are cleaved, tailored, and re-ligated in the form desired to generate the plasmids required. For analysis to confirm correct sequences in plasmids constructed, the ligation mixtures are used to transform host cells, and successful transformants selected by ampicillin or tetracycline resistance where appropriate. Plasmids from the transformants are prepared, analyzed by restriction endonuclease digestion, and/or sequenced.

Suitable host cells for cloning or expressing the DNA in the vectors herein are the prokaryote, yeast, or higher eukaryote cells, including without limitation plant, mammal, insect, etc. cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. These examples are illustrative rather than limiting.

In addition to prokaryotes, eukaryotic microbes such as filamentous fungi or yeast are suitable expression hosts. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as K. lactis, K. fragilis, etc.; Pichia pastoris; Candida; Neurospora crassa; Schwanniomyces such as Schwanniomyces occidentalis; and filamentous fungi such as Penicillium, Tolypocladium, and Aspergillus hosts such as A. nidulan, and A. niger.

Suitable host cells may also be derived from multicellular organisms. Such host cells are capable of complex processing and glycosylation activities. In principle, any higher eukaryotic cell culture is workable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa california NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can be utilized as hosts. Typically, plant cells are transfected by incubation with certain strains of the bacterium Agrobacterium tumefaciens. During such incubation of the plant cell culture, the DNA coding sequence is transferred to the plant cell host such that it is transfected, and will, under appropriate conditions, express the DNA. In addition, regulatory and signal sequences compatible with plant cells are available, such as the nopaline synthase promoter and polyadenylation signal sequences.

The mini-wnt polypeptides prepared by recombinant synthesis are typically isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Mini-wnt polypeptides of the present invention are usually biologically active in binding to a cognate Wnt receptor. In such instances, the mini-wnt specifically binds to a Wnt receptor and inhibits Wnt signaling when contacting a cell expressing the receptor, usually binding to one but not both or a Wnt co-receptor pair. One may determine the biological activity of a mini-wnt polypeptide in a composition by determining the amount of Wnt activity that is inhibited by the mini-wnt in a functional assay, e.g. destabilization of β-catenin, inhibition of growth of stem cells, etc., quantitating the amount of mini-wnt polypeptide present by a non-functional assay, e.g. immunostaining, ELISA, quantitation on coommasie or silver stained gel, etc., and determining the ratio of biologically active mini-wnt to total mini-wnt. An exemplary assay for mini-wnt polypeptide specific activity involves contacting cells with a Wnt-comprising composition, e.g. culture media from cells expressing Wnt protein, for a period of time sufficient to stabilize β-catenin, usually at least about 1 hour. A mini-wnt composition is then provided to the cells, and the cells are incubated, usually at least about an hour, to allow the mini-wnt polypeptide to competitively replace the Wnt protein. The cells are then lysed, and the cell lysate is resolved by SDS PAGE, then transferred to nitrocellulose and probed with antibodies specific for β-catenin. Other assays include C57MG transformation and induction of target genes in Xenopus animal cap assays. An effective dose or concentration of a mini-wnt polypeptide is that which will reduce signaling, for example as evidenced by the presence of nuclear β-catenin, by 25%, 50%, 75%, 90%, 95%, or more, relative to the signaling in the absence of the mini-wnt.

An aspect of the binding activity of mini-wnts is the ability to determine the receptor specificity of the full-length Wnt protein from which a mini-wnt is derived. Typically a mini-wnt used in such methods will have substantial sequence identity with the corresponding full-length Wnt protein, where the mini-wnt is usually identical to the corresponding full-length Wnt protein over at least a stretch of 50, 60, 70, 80, 90 or more contiguous amino acids. A major hindrance to confirming Wnt-Fz interactions has been the inability to express Wnts in order to measure their binding to Frizzled receptors. For example, see Kikuchi A, Yamet al. (2007) Cell Signal 19: 659-71; Logan and Nusse (2004) Annu Rev Cell Dev Biol 20: 781-810; and Wang et al. (2005) Mol Cell Biol 25: 5022-30. With mini-Wnts, which are easily expressed recombinantly and bind to the Fz-CRD, it is now possible in a straightforward way to match up Fz receptor with Wnts using the mini-Wnts as the binding reagents. To deorphanize Wnt-Fz interactions in this way is transformative for developmental as well as regenerative biology and drug design.

In methods for determining the cognate receptor for a Wnt protein, a candidate Wnt receptor or fragment thereof is contacted with a mini-wnt polypeptide that corresponds to a full-length Wnt protein of interest, which may be a native Wnt protein; and determining the binding of the mini-wnt to the candidate receptor, wherein the presence of specific binding is indicative that the full-length Wnt protein is a ligand for the candidate receptor. Receptors of interest include Fz protein, a ROR protein, or an Ryk protein, which may be contacted with a Cterm mini-wnt; and LRP5, LRP6, or FRL1/crypto, which may be contacted with an Nterm mini-wnt. Various binding assays find use, for example utilizing cells expressing the candidate receptor, but of particular interest are assays that can be performed in solution, e.g. utilizing a soluble fragment of the receptor for binding, including without limitation interactions between Fz-CRD polypeptides and water soluble Cterm mini-wnts.

In some embodiments, mini-wnt polypeptides of the present invention are conjugated to various functional moieties such as polypeptides, drugs, radionucleotides, or toxins. In other words, the subject composition comprises a functional moiety conjugated to the mini-wnt polypeptide. See, e.g., PCT publications WO 92/08495; WO 91/14438; WO 89/12624; U.S. Pat. No. 5,314,995; and EP 396,387.

One example of a functional moiety that may be conjugated to a mini-wnt polypeptide is a therapeutic moiety. Therapeutic moieties include, without limitation, moieties that promote cell death, and moieties that alter cellular activity.

Examples of moieties that promote cell death include cytotoxic agents, i.e. a cytotoxin, e.g., a cytostatic or cytocidal small molecule, a polypeptide agent or a radioactive metal ion. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicine, doxorubicin, daunorubicin, dihydroxy anthracenedione, mitoxantrone, mithramycin, actinomycin D, 1-dehydrotestosterone, glucocorticoids, procaine, tetracaine, lidocaine, propranolol, and puromycin and analogs or homologues thereof. Cytotoxic agents also include proteins, peptides, or polypeptides possessing a cytotoxic biological activity, e.g., toxins such as abrin, ricin A, pseudomonas exotoxin, cholera toxin, and diphtheria toxin. Cytotoxic agents also include radioactive metal ions, i.e. radionuclides, such as alpha-emitters, e.g. Bismuth-213, Radium-226, Lead-212, Actinium-225, and Astatine-211, and β-emitters, e.g. Iodide-131, Yttrium-90, Rhenium-188, Lutetium-177, Copper-67 and Copper-64, and macrocyclic chelators useful for conjugating radiometal ions, e.g. ¹³¹In, ¹³¹L, ¹³¹Y_{,} ¹³¹Ho, ¹³¹Sm, to polypeptides or any of those listed supra. Macrocyclic chelators can be attached to the antibody via a linker molecule, e.g. as described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Moieties that promote cell death also include moieties that target a cell for antibody-dependent cell-mediated cytotoxicity (ADCC), antibody dependent cell-mediated phagocytosis (ADCP), or complement dependent cytotoxicity (CDC, also known as complement-mediated cytolysis, or CMC), e.g. the Fc component of immunoglobulin. See, for example, Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). To assess ADCC activity of a molecule of interest, an in vitro ADCC assay may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). All FcγRs bind the same region on Fc, at the N-terminal end of the Cγ2 domain and the preceding hinge, which region may be utilized as a functional moiety for the purposes of the invention. An overlapping but separate site on Fc serves as the interface for the complement protein C1q. In the same way that Fc/FcγR binding mediates ADCC and ADCP, Fc/C1q binding mediates complement dependent cytotoxicity (CDC). A site on Fc between the Cγ2 and Cγ3 domains mediates interaction with the neonatal receptor FcRn, the binding of which recycles endocytosed antibody from the endosome back to the bloodstream

As used herein, an Fc fusion is synonymous with the terms “immunoadhesin”, “Ig fusion”, “Ig chimera”, and “receptor globulin” as used in the art (Chamow et al., 1996, Trends Biotechnol 14:52-60; Ashkenazi et al., 1997, Curr Opin Immunol 9:195-200). An Fc fusion combines the Fc region of an immunoglobulin with the Cterm or Nterm mini-wnt, for example. See for example U.S. Pat. Nos. 5,766,883 and 5,876,969, both of which are expressly incorporated by reference.

Therapeutic moieties other than those that promote cell death would include agents that alter the activity of a cell. Such therapeutic agents include, but are not limited to, cytokines, chemokines, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other functional moieties suitable for conjugation to subject mini-wnts of the present application include imaging moieties. As discussed above, an imaging moiety is a non-cytotoxic agent that can be used to locate and, optionally, visualize cells, e.g. cells that have been targeted by compositions of the subject application. For example, fluorescent dyes may be used as an imaging moiety. In another example, radioactive agents that are non-cytotoxic may also be an imaging moiety. An imaging moiety may require the addition of a substrate for detection, e.g. horseradish peroxidase (HRP), β-galactosidase, luciferase, and the like. Alternatively, an imaging moiety may provide a detectable signal that does not require the addition of a substrate for detection, e.g. a fluorophore or chromophore dye, e.g. Alexa Fluor 488® or Alexa Fluor 647®, or a protein that comprises a fluorophore or chromophore, e.g. GFP, RFP, dsRED, phiYFP, etc. and mutants thereof.

Functional moieties that induce multimers of two or more min-wnts are also of interest, e.g. including binding pairs having a high affinity, such as biotin and avidin/streptavidin, peptide sequences such as zipper domains, and the like, as known in the art.

Techniques for conjugating functional moieties to polypeptides are well known in the art, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Functional moieties are typically bound to the mini-wnt polypeptide of the subject compositions by covalent interactions. In some embodiments, a linker may be used, where the linker may be any moiety that can be used to link the mini-wnt polypeptide to the functional moiety. In some embodiments, the linker is a cleavable linker. The use of a cleavable linker enables the moiety linked to the mini-wnt polypeptide to be released from the mini-wnt polypeptide once absorbed by the cell, and transported to the cell body. The cleavable linker may be cleavable by a chemical agent, by an enzyme, due to a pH change, or by being exposed to energy. Examples of forms of energy that may be used include light, microwave, ultrasound, and radiofrequency.

In certain applications, it may be desirable to release the functional moiety, particularly where the moiety is a therapeutic moiety, once the compound has entered the cell, resulting in a release of the moiety. Accordingly, in one variation, the linker L is a cleavable linker. This enables the moiety M to be released from the compound once in a cell. This may be desirable when, for example, the functional moiety is a therapeutic moiety which has a greater therapeutic effect when separated from the mini-wnt polypeptide. For example, the therapeutic moiety may have a better ability to be absorbed by an intracellular component of the cell when separated from the mini-wnt polypeptide. Accordingly, it may be necessary or desirable to separate the therapeutic moiety from the mini-wnt polypeptide so that the therapeutic moiety can enter the intracellular compartment.

### Methods

In methods of the present invention, an effective amount of a composition comprising a mini-wnt is provided to cells, e.g. by contacting the cell with an effective amount of that composition to achieve a desired effect, e.g. to inhibit Wnt signaling, inhibit proliferation or aberrant angiogenesis, deliver a therapeutic or imaging moiety, generate an antibody, etc.

In some methods of the invention, an effective amount of the subject composition is provided to inhibit Wnt signaling in a cell. Biochemically speaking, an effective amount or effective dose of a Wnt inhibitor is an amount of inhibitor to decrease or attenuate Wnt signaling in a cell by at least 30%, at least 40%, at least 50%, at least 60%, at least 70%, at least 80%, at least 90%, at least 95%, or by 100% relative to the signaling in the absence of the mini-wnt. In other words, the responsiveness to Wnt signaling of a cell that has been contacted with an effective amount or effective dose of a mini-wnt composition will be about 70% or less, about 60% or less, about 50% or less, about 40% or less, about 30% or less, about 20% or less, about 10% or less, about 5% or less, or will be about 0%, i.e. negligible, relative to the strength of the response to Wnt signaling that is observed of a cell that has not been contacted with an effective amount/dose of a mini-wnt composition. The amount of modulation of a cell's activity by Wnt, that is, the responsiveness of a cell to Wnt signaling, can be determined by a number of ways known to one of ordinary skill in the art of Wnt biology. For example, the amount of phosphorylated β-catenin in a cell may be measured; the amount of cytosolic β-catenin in a cell may be measured; or the amount of activity of the transcription factors that are normally activated by Wnt signaling, e.g. TCF/LEF, may be measured, for example by measuring the RNA or protein levels of genes that are the transcriptional targets of TCF/LEF, or by transfecting/infecting the cell with a nucleic acid vector comprising a TCF binding site (TOP) operably linked to a reporter protein such as luciferase (TOPFlash), EGFP (TOP-EGFP), etc. and qualitatively or quantitatively measuring the amount of reporter protein that is produced. In this way, the antagonistic effect of the agent may be confirmed.

In a clinical sense, an effective dose of a mini-wnt composition is the dose that, when administered for a suitable period of time, usually at least about one week, and may be about two weeks, or more, up to a period of about 4 weeks, 8 weeks, or longer will evidence an alteration in the symptoms associated with undesired Wnt signaling. For example, an effective dose is the dose that when administered for a suitable period of time, usually at least about one week, and may be about two weeks, or more, up to a period of about 4 weeks, 8 weeks, or longer will slow or even halt tumor growth in a patient suffering from cancer or neovascularization in the eye of a patient suffering from diabetic retinopathy. In some embodiments, an effective dose may not only slow or halt the progression of the disease condition but may also induce the reversal of the condition. It will be understood by those of skill in the art that an initial dose may be administered for such periods of time, followed by maintenance doses, which, in some cases, will be at a reduced dosage.

In some embodiments, an effective amount of the subject composition is provided to deliver a functional moiety to a cell, e.g. a therapeutic moiety or an imaging moiety. In such embodiments, an effective amount will be the amount required to achieve therapeutic or imaging efficacy by the functional moiety.

For example, in some embodiments, the functional moiety is a therapeutic moiety that is cytotoxic. An effective amount of a composition comprising a cytotoxic moiety will be the amount sufficient to promote cell death selectively in the cells targeted by the mini-wnt composition to which the cytotoxic moiety is fused. In some instances, the effect amount of functional moiety is well known; e.g. radionuclides are typically delivered in the range of 10-30 cGy/h, the regimen depending on the half-life of the radioisotope. In other instances, the effective amount can be readily determined by one of ordinary skill in the art using any convenient method known in the art for assaying for cell death, e.g. TUNEL staining, Annexin staining, propidium iodide uptake, etc. It will be understood by those of skill in the art that an initial dose may be administered for such periods of time, followed by maintenance doses, which, in some cases, will be at a reduced dosage.

As another example, in some embodiments, the functional moiety is a therapeutic moiety that targets a cell for ADCC or CDC. An effective amount of a composition comprising a moiety that targets a cell for ADCC or CDC will be the amount sufficient to promote ADCC or CDC selectively in the cells targeted by the mini-wnt composition to which the cytotoxic moiety is fused. The effective amount can be readily determined by one of ordinary skill in the art using any convenient method known in the art for assaying ADCC and CDC.

As another example, in some embodiments, the functional moiety is an imaging moiety. The effective amount of a subject composition comprising an imaging moiety is the amount sufficient to selectively label the cells targeted by the mini-wnt composition to which the cytotoxic moiety is fused. The effective amount can be readily determined by one of ordinary skill in the art using any convenient method known in the art for visualizing imaging moieties, e.g. microscopy, e.g. epifluorescence or light microscopy.

The calculation of the effective amount or effective dose of mini-wnt composition to be administered is within the skill of one of ordinary skill in the art, and will be routine to those persons skilled in the art. Needless to say, the final amount to be administered will be dependent upon the route of administration and upon the nature of the disorder or condition that is to be treated.

Cells suitable for use in the subject methods are cells that comprise one or more Wnt receptors. As discussed above, Wnt receptors include Fz proteins, ROR proteins, Ryk, LRP5, LRP6 and EGF-CFC proteins. In some embodiments, the cell is a cell expressing a Wnt receptor comprising a CRD domain or a WIF domain. In such embodiments, the composition used in the method comprises a mini-wnt polypeptide that is a Cterm mini-wnt. Examples of Wnt receptors that comprise a CRD domain include Fz proteins and ROR transmembrane kinases. Examples of Wnt receptors that comprise a WIF domain include Derailed/Ryk. In some embodiments the cell is a cell expressing a Wnt receptor that is LRP5, LRP6, or crypto. In such embodiments, the composition used in the method comprises a mini-wnt polypeptide that is an Nterm mini-wnt.

The cells to be contacted may be in vitro, that is, in culture, or they may be in vivo, that is, in a subject. Cells may be from/in any organism, but are preferably from a mammal, including humans, domestic and farm animals, and zoo, laboratory or pet animals, such as dogs, cats, cattle, horses, sheep, pigs, goats, rabbits, rats, mice, frogs, zebrafish, fruit fly, worm, etc. Preferably, the mammal is human. Cells may be from any tissue. Cells may be frozen, or they may be fresh. They may be primary cells, or they may be cell lines. More usually, they are primary cells in vivo.

Cells of particular interest are those that are responsive to Wnt signaling and are associated with undesirable or otherwise aberrant cell proliferation, .e.g tumorigenesis, angiogenesis, etc. particularly as they may relate to Wnt-mediated disease conditions described below. As an example, cells of interest include endothelial cells, which are the cells that line the interior surface of blood vessels, and which, when aberrantly active, may be associated with aberrant angiogenesis. As another example, cells of interest include cancer cells, e.g. tumor cells, e.g. a cancer stem cell, which is a type of cancer cell that possesses characteristics associated with normal stem cells, namely the ability to give rise to all cell types found in a particular cancer sample, and which is associated with aberrant cell proliferation.

Cells in vitro may be contacted with a composition comprising a mini-wnt polypeptide by any of a number of well-known methods in the art. For example, the composition may be provided to the cells in the media in which the subject cells are being cultured. Nucleic acids encoding the mini-wnt polypeptide may be provided to the subject cells or to cells cocultured with the subject cells on vectors under conditions that are well known in the art for promoting their uptake, for example electroporation, calcium chloride transfection, and lipofection. Alternatively, nucleic acids encoding the Wnt inhibitor may be provided to the subject cells or to cells cocultured with the subject cells via a virus, i.e. the cells are contacted with viral particles comprising nucleic acids encoding the mini-wnt polypeptide. Retroviruses, for example, lentiviruses, are particularly suitable to the method of the invention, as they can be used to transfect non-dividing cells (see, for example, Uchida et al. (1998) P.N.A.S. 95(20):11939-44). Commonly used retroviral vectors are “defective”, i.e. unable to produce viral proteins required for productive infection. Rather, replication of the vector requires growth in a packaging cell line.

Likewise, cells in vivo may be contacted with the subject mini-wnt compositions by any of a number of well-known methods in the art for the administration of peptides or nucleic acids to a subject. The mini-wnt composition can be incorporated into a variety of formulations, which in some embodiments and particularly for Cterm mini-wnts will be formulated in the absence of detergents, liposomes, etc., as have been described for the formulation of full-length Wnt proteins. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the mini-wnt composition can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversibly affixed (see e.g. US Application Nos. 20080081064 and 20090196903, incorporated herein by reference).

Therapeutic Uses.

As alluded to above, mini-wnt compositions of the present invention find use in inhibiting Wnt signaling in a cell that is responsive to Wnt signaling. The responsiveness of a cell to a Wnt may be readily determined by one of ordinary skill in the art by methods known in the art and set forth herein. Biologically active mini-wnt compositions will inhibit, i.e. antagonize or suppress, Wnt signaling in a cell. Put another way, biologically active mini-wnt compositions are dominant negative regulators of Wnt signaling.

Dominant negative regulators of Wnt signaling such as the mini-wnt compositions of the present invention find use in the treatment of mammals, such as human patients, suffering from Wnt-mediated disease conditions such as disorders associated with aberrant cell proliferation or aberrant angiogenesis, including various cancers associated with Wnt. Patients suffering from diseases characterized by such conditions will benefit greatly by a treatment protocol of the pending claimed invention.

The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: colorectal cancer, chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), medulloblastoma and desmoid. Cancers of particular interest for treatment by the subject methods include gliomas, medulloblastomas, colon cancer, colorectal cancer, melanoma, breast cancer, lung cancer, liver cancer, and gastric cancer.

A composition comprising a mini-wnt polypeptide that inhibits the growth of a tumor is one which results in measurable reduction in the rate of proliferation of cancer cells in vitro or growth inhibition of a tumor in vivo. For example, preferred growth inhibitory Wnt antagonists will inhibit growth of tumor by at least about 5%, at least about 10%, at least about 20%, preferably from about 20% to about 50%, and even more preferably, by greater than 50% (e.g., from about 50% to about 100%) as compared to the appropriate control, the control typically being cancer cells not treated with the Wnt antagonist molecule being tested. The Wnt antagonist is growth inhibitory in vivo if administration of the Wnt antagonist at about 1 μg/kg to about 100 mg/kg body weight results in reduction in tumor size or cell proliferation within about 5 days to 3 months from the first administration, preferably within about 5 to 30 days.

As another example, compositions and methods of the present invention find use in inhibiting aberrant angiogenesis in a CNS cell. The term “angiogenesis” is used to describe the biological process by which new blood vessels grow or sprout from pre-existing vessels. Angiogenesis plays a critical role in the elaboration of vasculature both during embryogenesis and in the mature organism, for example, in wound healing. However, there are many disease states that are driven by persistent unregulated or improperly regulated angiogenesis. In such disease states, this aberrant angiogenesis may either cause a particular disease or exacerbate an existing pathological condition. For example, choroidal neovascularization (CNV) in the eye and the subsequent retinopathy has been implicated as the most common cause of blindness and underlies the pathology of a number of ocular diseases, most notably diabetic retinopathy and age related macular degeneration (AMD, ARMD), particularly wet/exudative age related macular degeneration. Wnt signaling has been implicated in promoting angiogenesis in the CNS and retina during development. Accordingly, Wnt inhibitors find use in treating—i.e. arresting the development or progression of—disease conditions of the CNS wherein aberrant angiogenesis is a contributing factor.

A mini-wnt composition that inhibits aberrant angiogenesis in the CNS or that inhibits neovascularization in the CNS is one which results in measurable inhibition of the development of new vasculature, for example tube formation by endothelial cells in culture or blood vessel formation in a subject. Preferred Wnt antagonists inhibit the rate of development of new vasculature by at least about 10%, at least about 20%, preferably from about 20% to about 50%, and even more preferably, by greater than 50% (e.g., from about 50% to about 100%) as compared to the appropriate control, the control typically being cells not treated with the Wnt antagonist molecule being tested. The Wnt antagonist is inhibitory in vivo if administration of the Wnt antagonist at about 1 μg/kg to about 100 mg/kg body weight results in a slowing or cessation of the development of neovasculature within about 5 days to 6 months from the first administration of the Wnt inhibitor, preferably within about 5 days to about 2 months. Neovasculature development may be observed by a number of ways that are well-known in the art and that will be obvious to the ordinary skilled artisan. For example, the inhibition of choroidal neovascularization may be readily observed directly by fundus photography, or indirectly by assaying for improved scoring on visual acuity tests.

Furthermore, other disorders are associated with aberrant Wnt signaling, including but not limited to osteoporosis, osteoarthritis, polycystic kidney disease, diabetes, schizophrenia, vascular disease, cardiac disease, non-oncogenic proliferative diseases, and neurodegenerative diseases such as Alzheimer's disease.

As an alternative to or in addition to inhibiting Wnt signaling, mini-wnt compositions may be used to deliver therapeutic moieties as discussed previously for the treatment of any of the aforementioned diseases. For example, mini-wnt compositions may be used to deliver cytotoxic moieties to tumorigenic cells, or to tag tumorigenic cells with Fc moieties that will target the cell for ADCC or CDC-mediated cell death. As another example, mini-wnt compositions may be used to delivery cytokines that promote synapse formation to neurons in neurodegenerative states, or axon outgrowth at sites of CNS injury. These and other therapeutic applications for the subject mini-wnt compositions will be readily apparent to the ordinarily skilled artisan.

For inclusion in a medicament, mini-wnt polypeptides may be obtained using generally accepted manufacturing methods. As a general proposition, the total pharmaceutically effective amount of the Wnt inhibitor compound administered parenterally per dose will be in a range that can be measured by a dose response curve.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The methods of the present invention also find use in combined therapies. For example, a number of agents may be useful in the treatment of aberrant angiogenesis, e.g. angiostatin, endostatin, VEGF inhibitors, etc. Likewise, a number of agents may be useful in the treatment of cancer, e.g. chemotherapeutic agents, radiotherapy, etc. The combined use of mini-wnts of the present invention and these other agents may have the advantages that the required dosages for the individual drugs is lower, and the effect of the different drugs complementary.

Delivery of Imaging Moieties.

As alluded to above, mini-wnt compositions of the present invention also find use in delivering imaging moieties to cells expressing Wnt receptors. For example, mini-wnt compositions conjugated to fluorescent moieties may be used to label and track cells in vitro and in vivo, e.g. for research purposes.

Antibody Generation.

Mini-wnt compositions of the present invention may also be used to generate antibodies. Antibodies may be generated by any suitable method known in the art. The antibodies of the present invention may be polyclonal or monoclonal antibodies. They may be monovalent, bivalent, or multivalent. They may be fragments, e.g. F(ab) fragments. Methods of preparing antibodies are known to the skilled artisan (Harlow, et al., Antibodies: a Laboratory Manual, (Cold spring Harbor Laboratory Press, 2nd ed. (1988), which is hereby incorporated herein by reference in its entirety).

In generating antibodies of the present invention, the subject composition comprising a mini-wnt polypeptide is formulated for injection, e.g. with an adjuvant, and the resulting immunogen is used to immunize animals. The mini-wnt polypeptide of an immunogen composition may, when beneficial, be produced as a fusion protein in which the mini-wnt polypeptide is attached to a fusion segment. The fusion segment often aids in protein purification, e.g., by permitting the fusion protein to be isolated and purified by affinity chromatography. Fusion proteins can be produced by culturing a recombinant cell transformed with a fusion nucleic acid sequence that encodes a protein including the fusion segment attached to either the carboxyl and/or amino terminal end of the protein. Fusion segments may include, but are not limited to, immunoglobulin Fc regions, glutathione-S-transferase, β-galactosidase, a poly-histidine segment capable of binding to a divalent metal ion, and maltose binding protein.

To generate polyclonal antibodies, the immunogen as described above may be administered to various host animals including, but not limited to, rabbits, mice, rats, etc., to induce the production of sera containing polyclonal antibodies specific for the antigen. The administration of the immunogen may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Additional examples of adjuvants which may be employed include the MPL-TDM adjuvant (monophosphoryl lipid A, synthetic trehalose dicorynomycolate). Immunization protocols are well known in the art in the art and may be performed by any method that elicits an immune response in the animal host chosen. Adjuvants are also well known in the art.

Typically, the immunogen (with or without adjuvant) is injected into the mammal by multiple subcutaneous or intraperitoneal injections, or intramuscularly or through IV. The immunogen may include an IL13 polypeptide, a fusion protein or variants thereof. Depending upon the nature of the polypeptides (i.e., percent hydrophobicity, percent hydrophilicity, stability, net charge, isoelectric point etc.), it may be useful to conjugate the immunogen to a protein known to be immunogenic in the mammal being immunized. Such conjugation includes either chemical conjugation by derivatizing active chemical functional groups to both the immunogen and the immunogenic protein to be conjugated such that a covalent bond is formed, or through fusion-protein based methodology, or other methods known to the skilled artisan. Examples of such immunogenic proteins include, but are not limited to, keyhole limpet hemocyanin, ovalbumin, serum albumin, bovine thyroglobulin, soybean trypsin inhibitor, and promiscuous T helper peptides. Various adjuvants may be used to increase the immunological response as described above.

Monoclonal antibodies may be prepared using hybridoma technology, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2.sup.nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies include, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo.

A variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hybridoma cells of the invention serve as a preferred source of such DNA. Once isolated, the DNA may be placed into expression vectors, which are then transformed into host cells such as NS0 cells, Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entirety.

Humanized antibodies are antibody molecules generated in a non-human species that bind the desired antigen having one or more complementarity determining regions (CDRs) from the non-human species and framework (FR) regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties). Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of Cole et al., and Boerder et al., are also available for the preparation of human monoclonal antibodies (Cole et al., Monoclonal Antibodies and Cancer Therapy, Alan R. Riss, (1985); and Boerner et al., J. Immunol., 147(1):86-95, (1991)). Human antibodies can also be produced using transgenic mice which are incapable of expressing functional endogenous immunoglobulins, but which can express human immunoglobulin genes.

Candidate mini-wnt antibodies may be tested by enzyme linked immunosorbent assay (ELISA), Western immunoblotting, or other immunochemical techniques to confirm their affinity and specificity for the target Wnt. Assays performed to characterize the individual antibodies include, but are not limited to (1) Inhibition of Wnt-autocrine proliferation of cancer stem cells; and (2) Inhibition of Wnt-induced TCF-LEF-induced gene expression.

Antibodies of the present invention may also be described or specified in terms of their cross-reactivity. Antibodies that bind mini-wnt polypeptides, which have at least 95%, at least 90%, at least 85%, at least 80%, at least 75%, at least 70%, at least 65%, at least 60%, at least 55%, and at least 50% identity (as calculated using methods known in the art and described herein) to human Wnts are also included in the present invention. Thus, antibodies of the present invention bind to the Wnt domain of the native parent Wnt protein from which the mini-wnt was derived and inhibit the activation of the receptor complexes that are normally bound by that Wnt.

Preferred binding affinities include those with an equilibrium dissociation constant or K_{D} from 10⁻⁸ to 10⁻¹⁵ M. The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

We sought to identify water-soluble versions of Wnt or Wnt fragments that retained biological activity by using in vitro evolution methods. Given the difficulties posed by wild-type Wnts in that they are decorated with lipid groups that greatly reduce their solubility and ability to be expressed recombinantly, we reasoned that water soluble Wnts or Wnt fragments that retained receptor binding activity would be transformative for Wnt studies on every level: biochemical, functional, and translational for design of Wnt drugs either as antagonists or agonists.

Towards this end, in vitro evolution was used to discover forms of Wnt that are a) active at binding the receptor Frizzled, and b) water soluble. Yeast surface display was used to screen Wnt variants for those that meet these criteria. Because yeast do not possess the requisite enzymes to attach the lipid groups to Wnt, the yeast surface display system is uniquely suited for this purpose: any mutant Wnt that is identified as having a binding affinity for Fz must, because it is expressed by yeast in the screen, be devoid of lipid groups and hence be water soluble. As proof of concept, no binding to a Fz-CRD construct was observed following expression of the wild-type sequence of Xenopus XWnt8 on yeast.

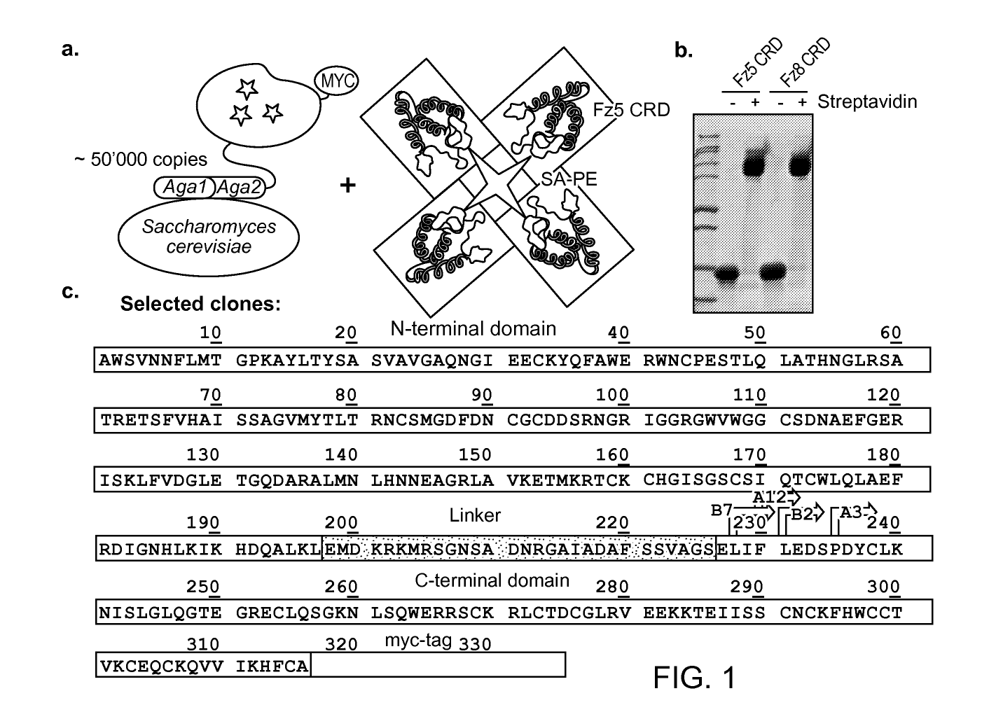

To identify mutants or variant forms of XWnt8 that could be expressed by yeast and bind correctly to the Fz-CRD, a large library of mutated XWnt8 genes was generated using error-prone PCR, which introduces 3-4 bases change per gene. These variants were then fused the yeast Aga2 protein, and the fusion constructs transformed into yeast. The library of Wnt variants (˜50,000 copies per yeast) was screened using a Fz-CRD (in this case Fz5-CRD) composition in which the Fz-CRD was conjugated to streptavidin to form strep-Fz-CRD “tetramers” (FIG. 1). Conjugation to streptavidin raised the avidity, or multimeric nature, of the Fz-CRD, thereby enhancing the sensitivity of the reagent. For example, as demonstrated in FIG. 1b, Fz5 and Fz8 CRD are completely biotinylated, since the addition of streptavidin forces the CRDs (now bound by streptavidin, see the ‘+” lanes) to run at a much higher molecular weights. Thus strep-Fz-CRD “tetramers” are very efficient reagents to use for the yeast library selection.

The color coding of the gene and designation and N-terminal domain, Linker, and C-terminal domain was done in hindsight in this figure based on the experiments shown in subsequent figures. Prior to the selection experiments, it was not known that the Wnt gene was subdivided into these regions, and it is only in view of the results of the in vitro evolution experiments that these boundaries can be drawn on the Wnt gene. The red arrows on the gene in FIG. 1C designate several of the boundaries for truncated mini-Wnt C-terminal fragments to show what proportion the mini-Wnts encompass of the full-length gene.

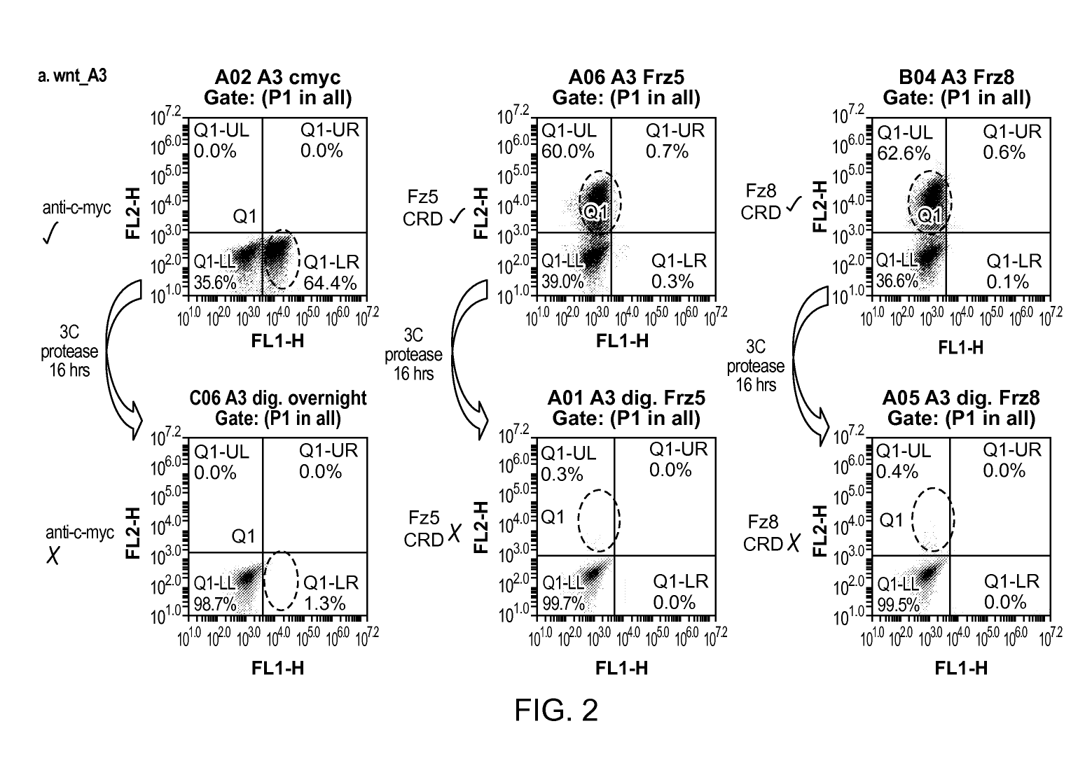

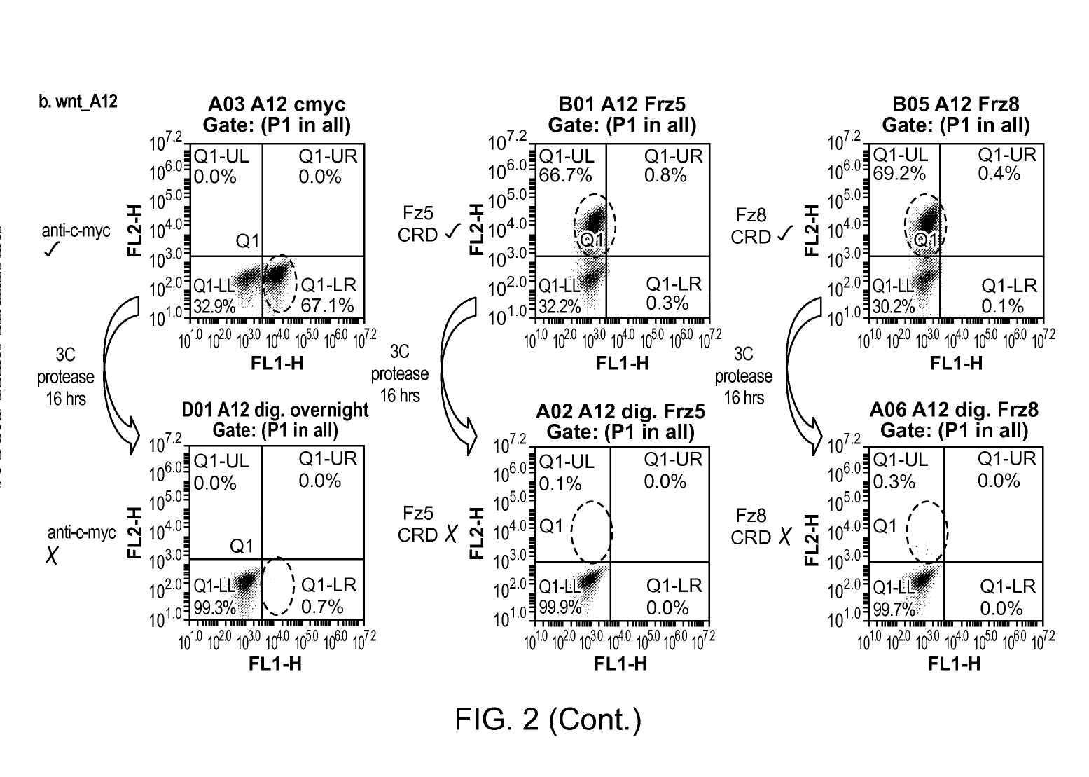

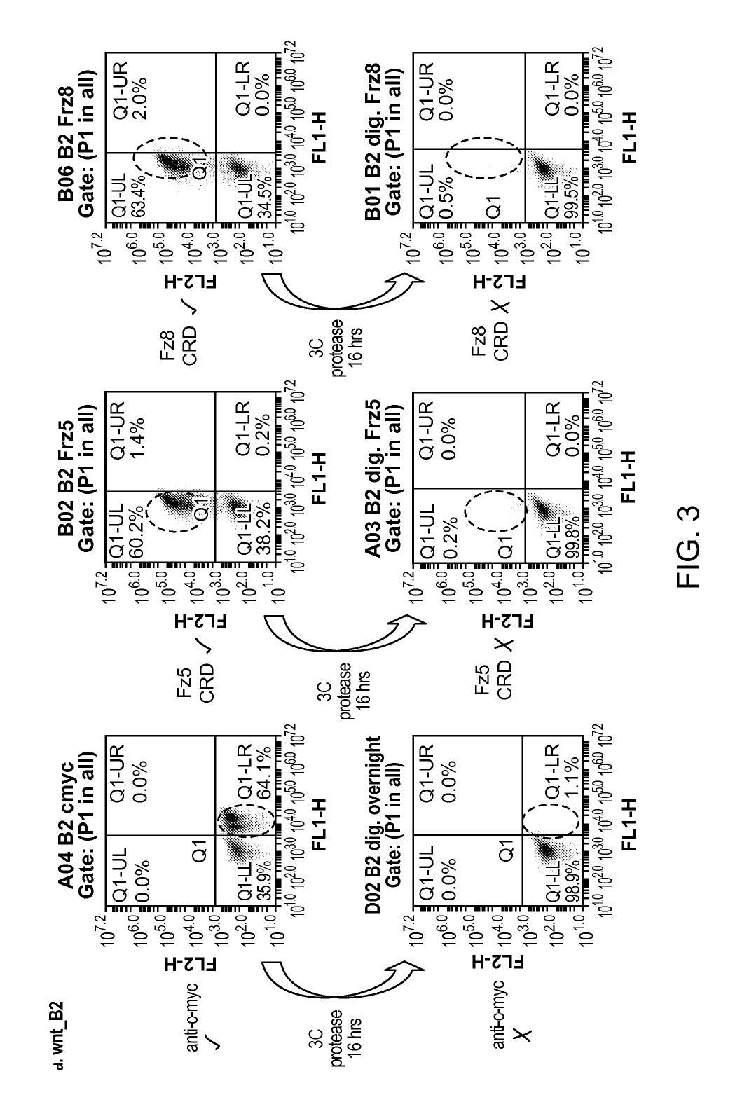

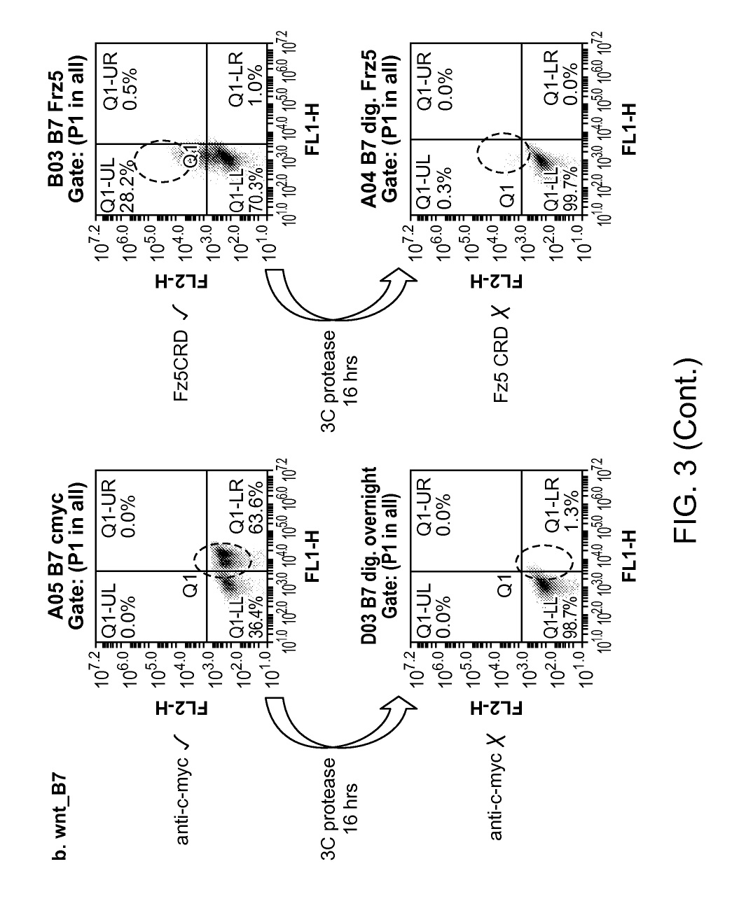

After several rounds of selecting the XWnt8 error prone libraries on Fz5 and Fz8-CRD tetramers individual yeast clones were isolated that appeared to be specific and reactive. The specificity of individual yeast clones for the Fz-CRD tetramers was confirmed by FACS analysis. Briefly, each fusion construct was designed to include a protease site in the linker between the yeast and the XWnt8, and a C-terminal Myc tag at the end of the XWnt8 on yeast. By staining the yeast with Fz-CRD tetramers or Myc-specific antibody in the absence and presence of the protease, it was robustly determined if the tetramers were, in fact, specifically reacting with the Wnt, or if the interaction was merely a non-specific binding to the yeast. As shown in FIGS. 2 and 3, the FACS staining by the Fz-CRD tetramers is lost when protease in added, indicating that reactivity of the yeast clone for the Fz-CRD was, indeed, due to the Wnt variant that was displayed by that yeast clone and not a non-specific affinity of the Fz-CRD for the yeast. Similarly, in the presence of the protease, Myc reactivity is lost. Thus, a specific binding interaction was confirmed between the yeast clones identified and FzCRD.

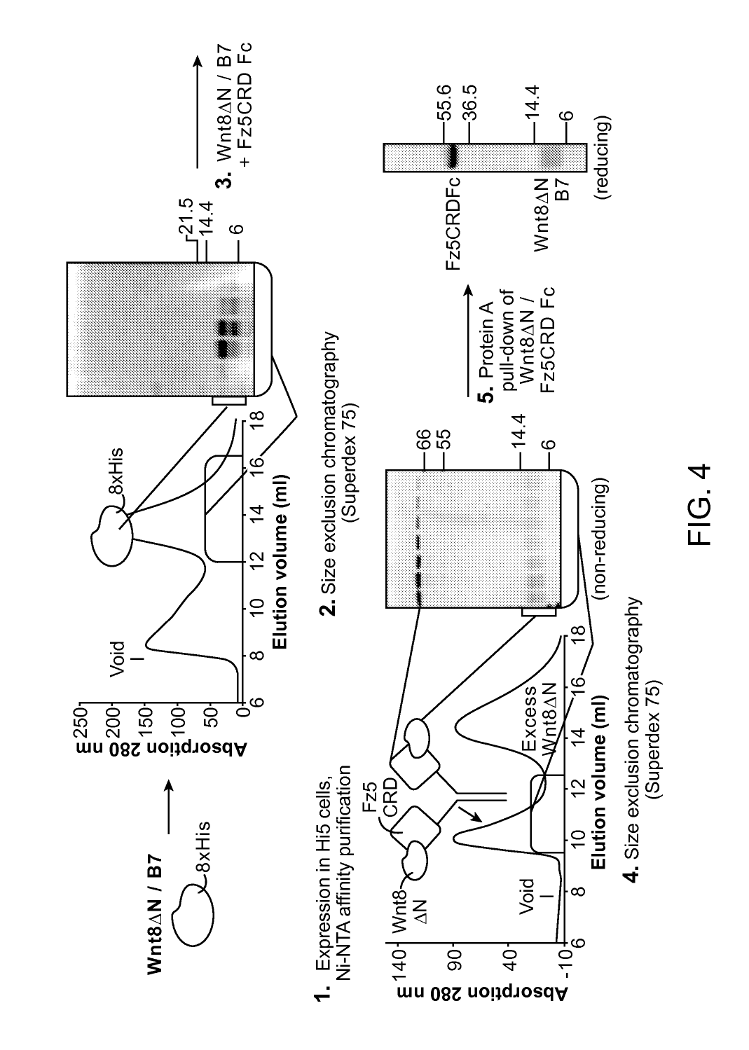

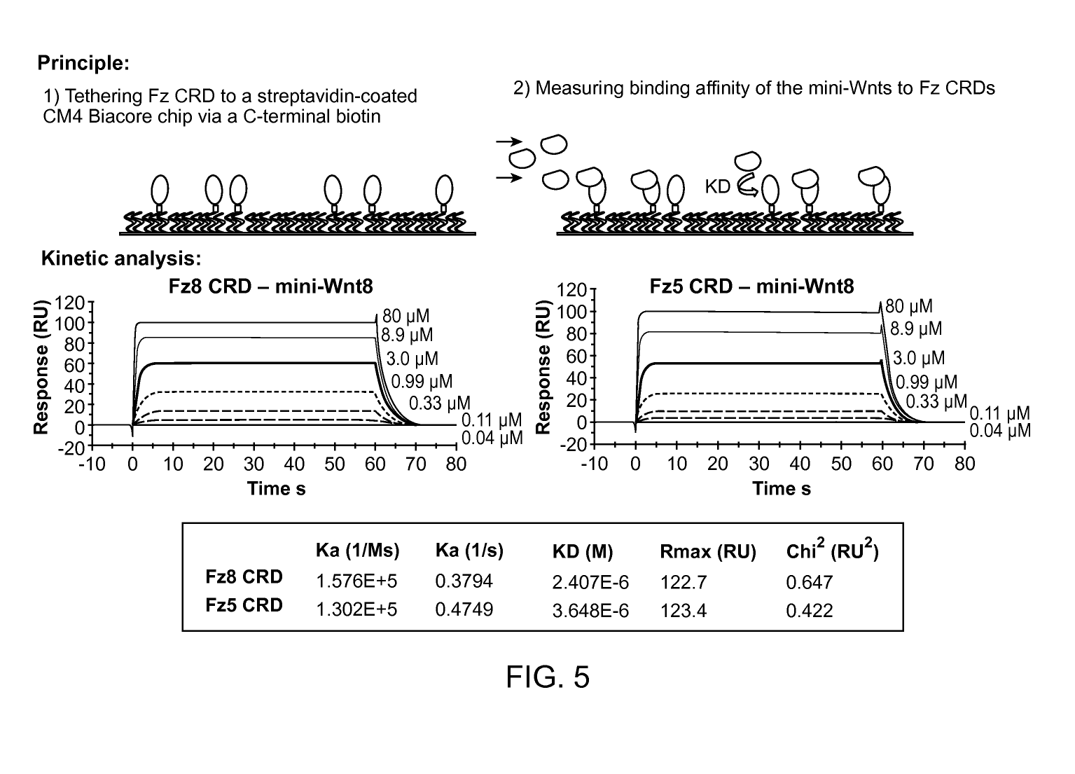

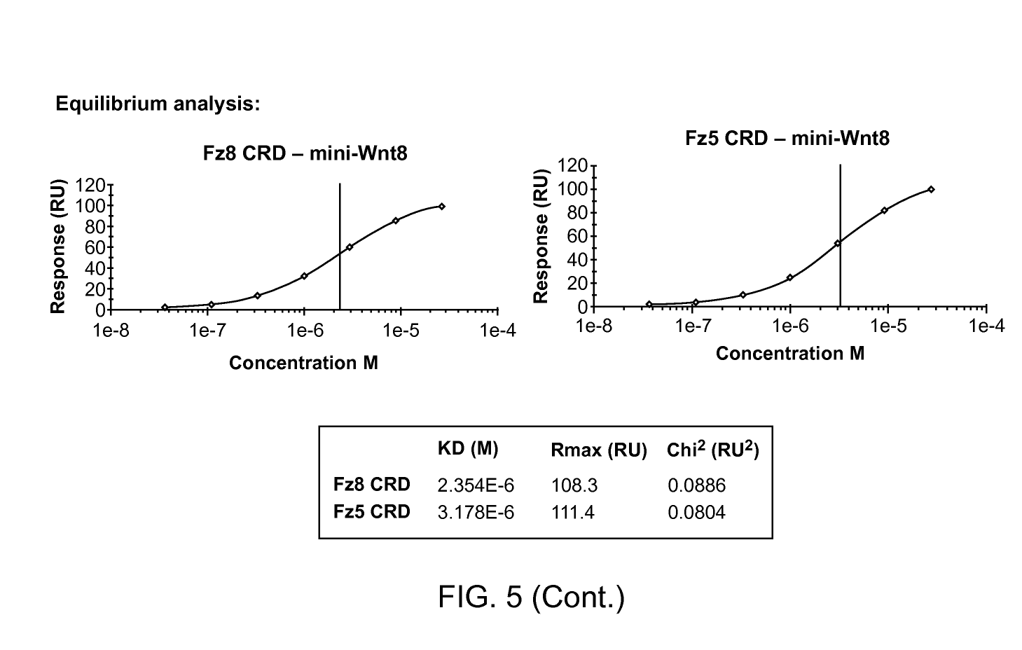

Yeast clones expressing XWnt8 variants which demonstrated a specific binding activity for Fz-CRD were expressed in a soluble recombinant form (FIG. 4). As one example, the XWnt8 variant B7 was expressed in insect cells and purified by gel filtration, which shows it to be a well folded and water soluble protein in the absence of detergent. (FIG. 4, top). Fz5-CRD added to this purified B7 variant co-elute with the B7 variant as a complex by gel filtration, demonstrating that the Fz5-CRD “pulls down” or specifically binds to this recombinant water soluble XWnt8 variant. Thus, the XWnt8 variant B7 is a water-soluble and receptor binding version of Wnt.

The analysis of additional XWnt8 variants as described above revealed that all of the variants that were soluble and bound to Fz-CRD are small, truncated versions of the full-length XWnt8. These sequences only encompassed the C-terminal ˜100 amino acids or so of Wnt, and thus, were dubbed “mini-Wnts.” While the exact boundaries of the mini-Wnt can vary (as shown in FIG. 1) as long as the principal region (pink in FIG. 1) is encompassed, these mini-wnts retain Fz binding activity. Since this region is highly conserved in all Wnts across species, the mini-Wnt represents a conserved Fz binding fragment of Wnts across all species.

To accurately quantify the binding affinity of the mini-wnt variants for the Fz-CRD, surface plasmon resonance (SPR) was performed. MiniXWnt8 was expressed and purified from baculovirus. Biotinylated Fz5- and Fz8-CRD was coupled to a SPR chip, over which was flowed baculovirus-expressed mini-Xwnt8. In order to generate a binding curve that is fittable to mathematical models, several mini-Wnt concentrations were injected over the chip and the extent of binding recorded in Response Units (RU). From this data we were able to fit binding curves and calculate affinity constants (KD) of −1-2 micromolar. Thus, this experiment proves in a purified system that mini-XWnt8 binds to Fz-CRD with physiological affinities in aqueous buffers lacking detergent.

The relevance of the discovery of the mini-XWnt8 to other Wnts is very clear. FIG. 6 shows a sequence alignment of all human Wnts together with the XWnt8 encompassing the mini-Wnt region of Wnt, approximately the last 110 residues of Wnt. The sequences are highly conserved, indicating that the Fz binding domain of all mammalian, vertebrate, and invertebrate Wnts resides in the C-terminal mini-Wnt domain thus delineated. This domain does not contain any lipid addition sites and is water soluble, and thus provides an ideal platform from which to produce Wnt polypeptides that modulate Wnt signaling.

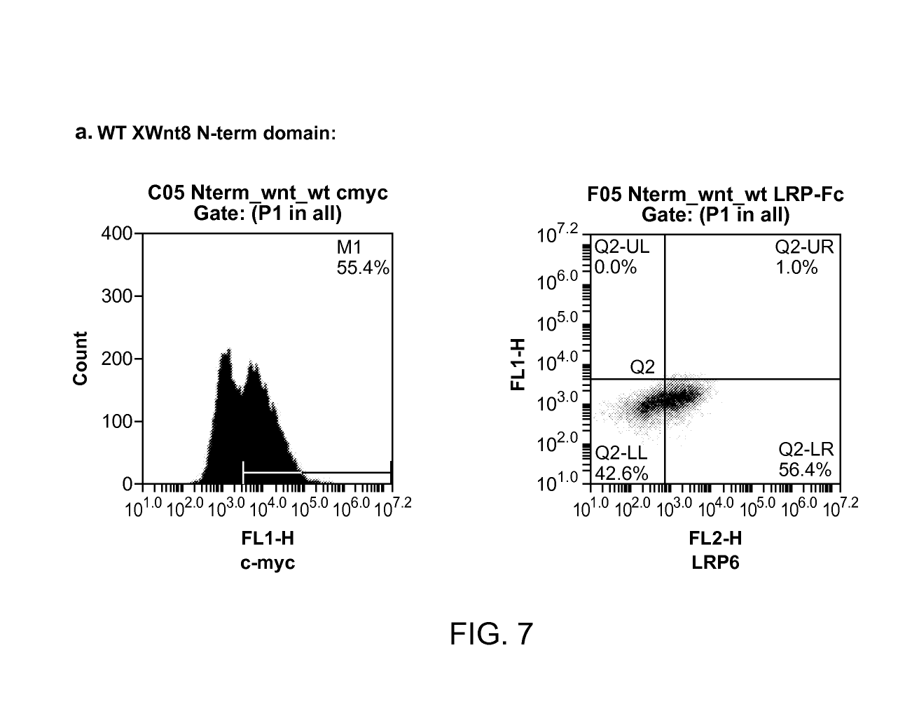

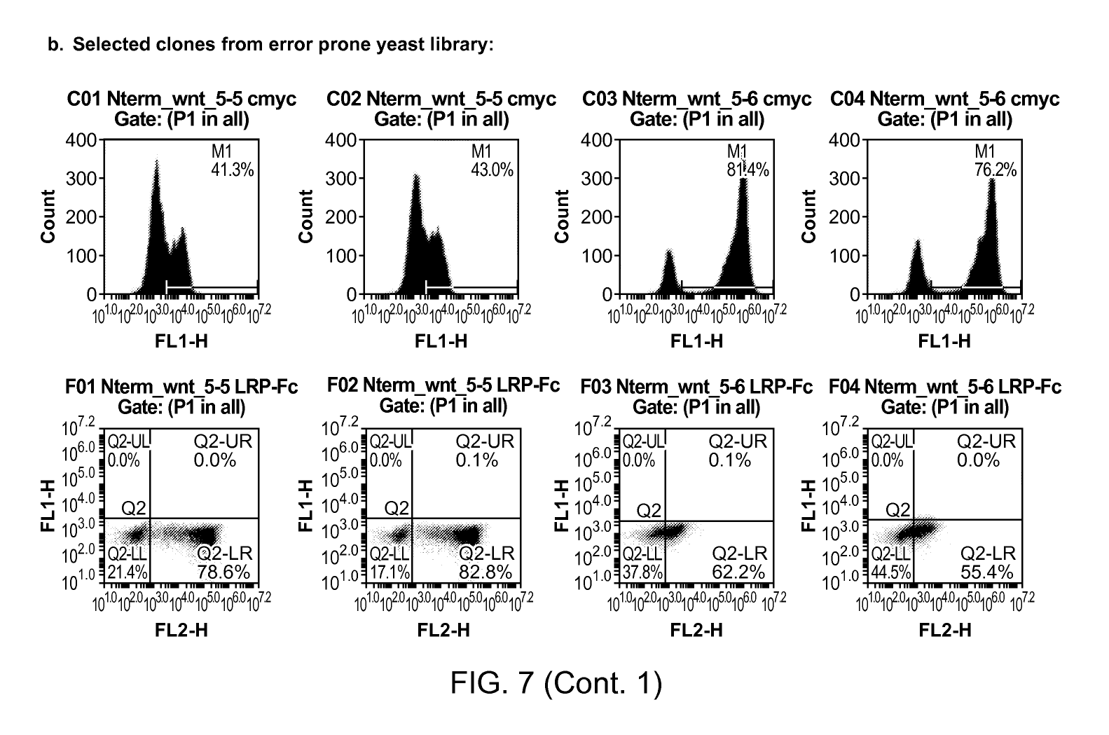

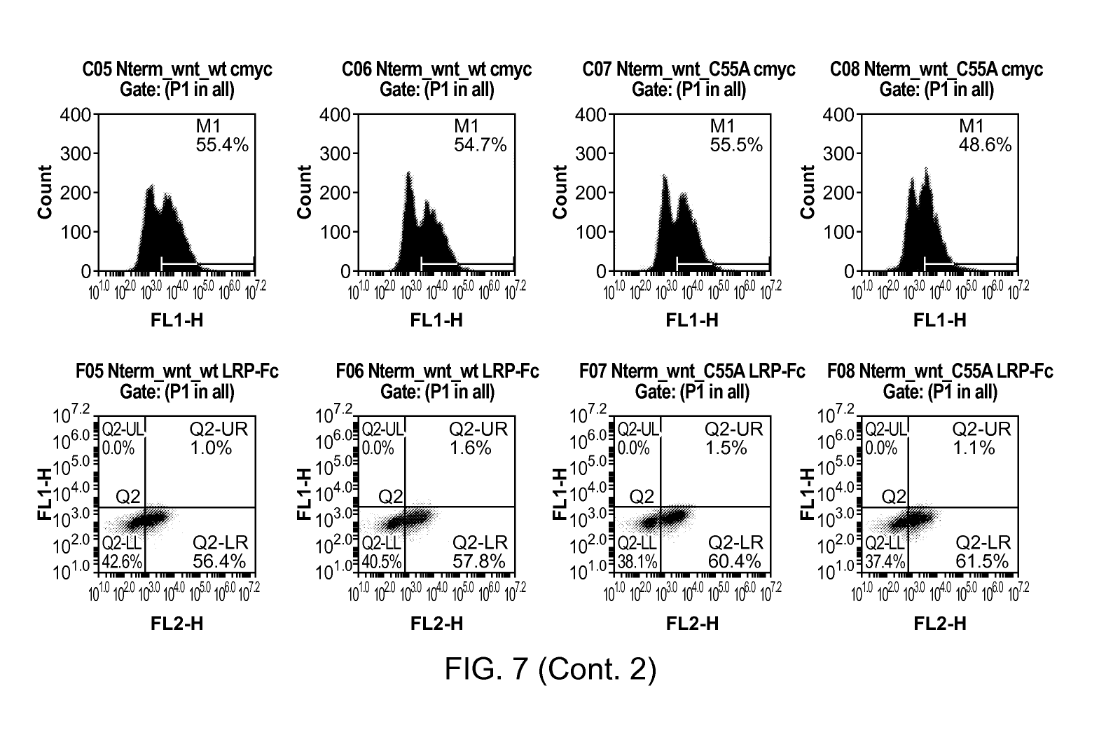

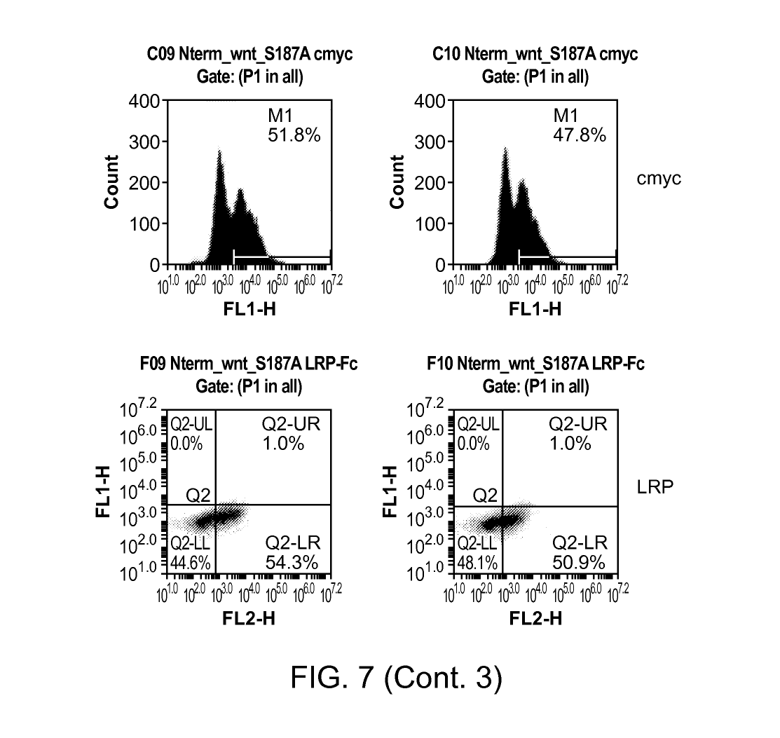

It is known that most Wnt signaling through the Fz receptor requires Wnt binding to both Fz and a co-receptor, e.g. LRP5/6. However, nothing is known about the domains of Wnt necessary for this co-receptor interaction. Based on our discover that mini-Wnt C-terminal domain binds to Fz, we reasoned that the N-terminal domain (as shown in FIG. 1) binds to Lrp6. To determine if the N-terminal domain of Wnt interacts with Lrp6, similar experiments using yeast display were performed as above. First, yeast clones expressing the wild-type XWnt8 N-terminal domain were shown to stain with the Myc antibody, proving that the N-terminal mini-Wnt is expressed on the surface of yeast (FIG. 7a, left panel). Binding of the LRP6 to the wild-type XWnt8 on yeast was then demonstrated, clearly proving that the N-terminal domain of mini-Wnt is the Lrp6 binding domain (FIG. 7a, right panel).

We then created an error prone library of the XWnt8 N-terminal domain and selected clones using LRP6 in an analogous approach to the Cterm mini-wnts. FIG. 7b shows a range of yeast clones from this error prone library that stain specifically with the myc antibody and with the Lrp6 receptor. Several of these clones stain much more strongly than wild-type N-terminal domain and may therefore be more stable or bind with higher affinity. Thus, FIG. 7 provides evidence for the creation of a biologically active mini N-terminal Wnt. In FIG. 8, we show that the N-terminal region of human Wnts is also very conserved and that these forms of Wnt also use the N-terminal domain to bind to Lrp6. Thus, while our studies used Xenopus Wnt8, the results are extrapolated to other species given the strong sequence conservation.

Collectively, these experiments demonstrate that Wnt is divided into an N-terminal Lrp5/6 binding domain and a water soluble C-terminal mini-Wnt Fz binding domain. Either domain can be used as platform to create Lrp5/6 or Fz binding molecules that serve as diagnostic agents or as therapeutic agents for modulating Wnt signaling.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A composition comprising a mini-wnt polypeptide, wherein the mini-wnt polypeptide is either a Cterm mini-wnt polypeptide or an Nterm mini-wnt polypeptide.

2. The composition of claim claim 1, wherein the mini-wnt polypeptide is a Cterm mini-wnt polypeptide that binds to a Fz protein, a ROR protein, or an Ryk protein and does not bind to a corresponding Wnt co-receptor.

3. The composition of claim 2, wherein the Cterm mini-wnt polypeptide consists of a Cterm mini-wnt amino acid sequence as set forth in FIG. 6.

4. The composition of claim 2, wherein the Cterm mini-wnt polypeptide aligns by conserved residues with positions 298-370 of human Wnt1 and lacks the amino acid sequence that aligns with residues 1-257 of human Wnt1.

5. The composition of claim 1, wherein:
the mini-wnt polypeptide is an Nterm mini-wnt polypeptide that binds to an LRP5, LRP6, or FRL1/crypto protein, and does not bind to a corresponding Wnt co-receptor.

6. The composition of claim 5, wherein the Nterm mini-wnt polypeptide consists of a Wnt amino acid sequence as set forth in FIG. 8.

7. The composition of claim 5, wherein the Nterm mini-wnt polypeptide aligns by conserved residues with positions 34-247 of human Wnt1 and lacks the amino acid sequence that aligns with residues corresponding to residues 288-370 of human Wnt1.

8. The composition according to claim 1, wherein the variant is a truncated form.

9. The composition according to claim 1, wherein the variant comprises one or more amino acid deletions or substitutions.

10. The composition according to claim 1, wherein the mini-wnt polypeptide is water soluble.

11. The composition according to claim 1, further comprising a fused or conjugated functional moiety.

12. The composition according to claim 11, wherein the functional moiety is a therapeutic moiety or an imaging moiety.

13. The composition according to claim 1, wherein the composition is formulated with an adjuvant.

14. The composition according to claim 1, wherein the composition is formulated for pharmaceutical administration.

15. A method for inhibiting Wnt signaling in a cell, comprising:
contacting a cell expressing a Wnt receptor with an effective amount of a composition as set forth in claim 1, wherein Wnt signaling is inhibited.

16. The method according to claim 15, wherein proliferation of the cell is inhibited.

17. The method according to claim 16, wherein the cell is a cancer cell.

18. The method according to claim 15, wherein the cell is in vitro.

19. The method according to claim 15, wherein the cell is in vivo.

20. A method of delivering a functional moiety to a cell, the method comprising: contacting a cell expressing a Wnt receptor with a composition as set forth in claim 11 or claim 12.

21. A method of determining the cognate receptor for a Wnt protein, the method comprising:
contacting a candidate Wnt receptor or fragment thereof with a mini-wnt polypeptide that corresponds to a Wnt protein of interest;
determining the binding of the mini-wnt polypeptide to the candidate receptor;
wherein the presence of specific binding is indicative that the Wnt protein is a ligand for the candidate receptor.
